(19) United States
(12) Patent Application Publication
(10) Pub. No.: US 2023/0131370 A1
(43) Pub. Date: Apr. 27, 2023

(54) AN UNMANNED AERIAL VEHICLE (UAV)-BASED SYSTEM FOR COLLECTING AND DISTRIBUTING ANIMAL DATA FOR MONITORING

(71) Applicant: SPORTS DATA LABS, INC., Royal Oak, MI (US)

(72) Inventors: Mark GORSKI, Royal Oak, MI (US); Vivek KHARE, Cupertino, CA (US); Stanley MIMOTO, Bethel Island, CA (US)

(73) Assignee: SPORTS DATA LABS, INC., Royal Oak, MI (US)

(21) Appl. No.: 16/977,570

(22) PCT Filed: Jul. 20, 2020
(86) PCT No.: PCT/US2020/042705

§ 371 Date: Sep. 2, 2020

Related U.S. Application Data
Application No. PCT/US20/42705 is a continuation-in-part of application No. 16/517,012, filed on Jul. 19, 2019, now Pat. No. 10,980,218.

(51) Int. Cl. A01K 29/00; B64C 39/02; G16H 50/20

(52) U.S. Cl. CPC A01K 29/005; B64C 39/024; G16H 50/20; B64C 2201/122

(57) ABSTRACT

An unmanned aerial vehicle-based data collection and distribution system includes a source of animal data that can be transmitted electronically. The source of animal data includes at least one sensor. The animal data is collected from at least one target individual. The system also includes an unmanned aerial vehicle that receives the animal data from the source of animal data as a first set of received animal data and a home station that receives the first set of received animal data. Characteristically, the unmanned aerial vehicle includes a transceiver operable to receive signals from the source of animal data and to send control signals to the source of animal data.

### TECHNICAL FIELD

In at least one aspect, the present invention is related to collecting and distributing animal data via one or more unmanned aerial vehicles.

### BACKGROUND

The continuing advances in the availability of information over the Internet have substantially changed the way that business is conducted. Simultaneous with this information explosion, sensor technology, and moreover, biosensor technology has also progressed. In particular, miniature biosensors that measure electrocardiogram signals blood flow, body temperature, perspiration levels or breathing rate are now available. Centralized service providers that collect and organize information collected from such biosensors to monetize such information do not exist. Moreover, access to and monitoring such sensors while individuals are in a designated location, moving horn one location to another, or engaged in activities that necessitate monitoring in dynamic and mobile environments such as sports and geneial fitness present issues regarding accessibility which are unknown.

Accordingly, there is a need for systems that collect and organize sensor data from an individual or group of individuals during activities that require monitoring.

### SUMMARY

In at least one aspect, an unmanned aerial vehicle-based data collection and distribution system is provided. The unmanned aerial vehicle-based data collection and distribution system includes a source of animal data that is electronically transmittable. The source of animal data includes at least one sensor. The animal data is collected from at least one targeted individual. The system also includes an unmanned aerial vehicle that receives the animal data from the source of animal data as a first set of received animal data, and a computing device that is operable to receive at least a portion ol the first set of received animal data. Characteristically, the unmanned aerial vehicle includes a transceiver operable to receive one or more signals from the source of animal data and to send one or more control signals to the source of animal data.

In at least another aspect, an unmanned aerial vehicle-based data collection and distribution system is provided. The unmanned aerial vehicle-based data collection and distribution system includes one or more sources of animal data that are electronically transmittable. The one or more sources of animal data include at least one sensor. The animal data is collected from at least one targeted individual. The system also includes one or more unmanned aerial vehicles that receive the animal data from the one or more sources of animal data as a first set of received animal data, and one or more computing devices that are operable to receive at least a portion of the first set of received animal data. Characteristically, the unmanned aerial vehicle includes a transceiver operable to receive one or more signals from the one or more sources of animal data and to send one or more control signals to the one or more sources of animal data.

Advantageously, the methods and systems set forth herein have applications in sports/fitness, general health & wellness monitoring, military operations & training, risk mitigation industries (e.g., insurance), and the like.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1 is a schematic of an unmanned aerial vehicle-based collection and distribution system using one or more sensors to acquire sensor data fiom a targeted individual.

FIG. 2 is a schematic of an unmanned aerial vehicle-based collection and distribution system using one or more sensors to acquire sensor data from multiple targeted individuals.

FIG. 3 is a schematic of an unmanned aerial vehicle with an integrated computing device.

FIGS. 4A, 4B, and 4C are illustrations of a user interface for operation of the unmanned aerial vehicle based collection and distribution system.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims the singular form “a,” “an,” and “the” comprise plural references unless the context clearly indicates otherwise. For example, a reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingiedient not specified in the claim. When this phrase appears in a clause ot the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” In addition, the term “plurality” means “multiple” and the term “multiple” means “plurality.” The terms “one or more” and “at least one” include “plurality” and “multiple” as a subset. In a refinement, “one or more” includes “two or more.”

Throughout this application, where publications are referenced, the disclosures of these publications in their entireties are hereby incorporated by reference into this application to more fully describe the state of the art to which this invention pertains.

When a computing device is described as performing an action or method step, it is understood that the computing device is operable to perform the action or method step typically by executing one or more lines of source code. The actions or method steps can be encoded onto non-transitory memory (e.g., hard drives, optical drive, flash drives, and the like).

The term “computing device” generally refers to any device that can perform at least one function, including communicating with another computing device. In a refinement, a computing device includes a central processing unit that can execute program steps and memory for storing data and a program code.

The term “server” refers to any computer or other computing device (including, but not limited to, desktop computer, notebook computer, laptop computer, mainframe, mobile phone, smart watch/glasses, augmented reality headset, virtual reality headset, and the like), distributed system, blade, gateway, switch processing device, or a combination thereof adapted to perform the methods and functions set forth herein.

The term “connected to” means that the electrical components referred to as connected to are in electrical or electronic communication In a refinement, “connected to” means that the electrical components referred to as connected to are directly wired to each other. In another refinement, “connected to” means that the electrical components communicate wirelessly or by a combination of wired and wirelessly connected components. In another refinement, “connected to” means that one or more additional electrical components are interposed between the electrical components referred to as connected to with an electrical signal from an originating component being processed (e.g, filtered, amplified, modulated, rectified, attenuated, summed, subtracted, etc.) before being received to the component connected thereto.

The term “electrical communication” or “electronic communication” means that an electrical signal is either directly or indirectly sent from an originating electronic device to a receiving electrical device. Indirect electrical or electronic communication can involve processing of the electrical signal, including but not limited to, filtering of the signal, amplification of the signal, rectification of the signal, modulation of the signal, attenuation of the signal, adding of the signal with another signal, subtracting the signal from another signal, subtracting another signal from the signal, and the like. Electrical or electronic communication can be accomplished with wired components, wirelessly connected components, or a combination thereof.

The processes, methods or algorithms disclosed herein can be deliverable to implemented by a controller, computer, or other computing device which can include any existing programmable electronic control unit or dedicated electronic control unit. Similarly, the processes, methods, or algorithims can be stored as data and instructions executable by a controller, computer, or computing device in many forms including, but not limited to, information permanently stored on nonwritable storage media such as ROM devices and information alterably stored on writeable storage media such as floppy disks, magnetic tapes, CDs, RAM devices, other magnetic and optical media, and shared or dedicated cloud computing resources. The processes, methods, or algorithms can also be implemented in an executable software object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software, and firmware components.

The terms “subject” and “individual” are synonymous and refer to a human or other animal, including birds, reptiles, amphibians, and fish, as well as all mammals including primates (particularly higher primates), horses, sheep, dogs, rodents, pigs, cats, rabbits, and cows. The one or more subjects may be, for example, humans participating in athletic training or competition, horses racing on a race track, humans playing a video game, humans monitoring their personal health, humans providing their data to a third party, humans participating in a research or clinical study, or humans participating in a fitness class. In a refinement, the subject or individual can be one or more machines (e.g., robot, autonomous vehicle mechanical arm) or networks of machines programmable by one or more computing devices that share at least one biological function with a human or other animal and from which one or more types of biological data can be derived, which may be, at least in part, artificial in nature (e.g., data from artificial intelligence-derived activity that mimics biological brain activity).

The term “animal data” refers to any data obtainable from, or generated directly or indirectly by, a subject that can be transformed into a form that can be transmitted (e.g., wireless or wired transmission) to a server or other computing device. Animal data includes any data, including any signals or readings, that can be obtained from sensors or sensing equipment systems and in particular, biological sensors (biosensors). Animal data an also include any descriptive data related to a subject, auditory data related to a subject, data that can be manually entered related to a subject (e.g., medical history, social habits, feelings of a subject), and data that includes at least a portion of real animal data. In a refinement, the term “animal data” is inclusive of any derivative of animal data. In another refinement, animal data includes at least a portion of simulated data. In yet another refinement animal data is inclusive of simulated data.

The term “sensor data” refers to both the unprocessed and/or processed (e g. manipulated) signal or reading generated by a sensor. In some cases, the term sensor data may also include metadata associated with the sensor or the one or more signals or readings (e.g., characteristics).

The term “artificial data” refers to artificially-created data that is derived from, based on, or generated using, at least in part, real animal data or its one or more derivatives. It can be created by running one or more simulations utilizing one or more artificial intelligence techniques or statistical models, and can include one or more signals or readings from one or more non-animal data sources as one or more inputs. Artificial data can also include any artificially-created data that shares at least one biological function with a human or another animal (e.g., artificially-created vision data, artificially-created movement data). It is inclusive of “synthetic data,” which can be any production data applicable to a given situation that is not obtained by direct measurement. Synthetic data can be created by statistically modeling original data and then using those models to generate new data values that reproduce at least one of the original data’s statistical properties. For the purposes of the presently disclosed and claimed subject matter, the terms “simulated data” and “synthetic data” are synonymous and used interchangeably with “artificial data,” and a reference to any one of the terms should not be interpreted as limiting but rather as encompassing all possible meanings of all the terms.

The term “insight” refers to one or more descriptions that can be assigned to a targeted individual that describe a condition or status of the targeted individual utilizing at least a portion of their animal data. Examples include descriptions or other characterizations of stress levels (e.g high stress, low stress), energy levels fatigue levels, and the like. An insight may be quantified, characterized, or communicated by one or more numbers, codes, graphs, charts, lots colors, or other visual representations, plots, readings, numerical representations, descriptions, text, physical responses, auditory responses, visual responses, kinesthetic responses, or verbal descriptions that are predetermined. In a refinement, an insight is comprised of a plurality of insights. In another refinement, an insight can be assigned to multiple targeted individuals, as well as one or more groups of targeted individuals. In another refinement, an insight can include one or more signals or readings from non-animal data sources as one or more inputs in its one ot more calculations, computations, derivations, incorporations, simulations, extractions, extrapolations, modifications, enhancements, creations, estimations, deductions, inferences, determinations, processes, communications, and the like.

The term “computed asset” refers to one or more numbers, a plurality of numbers, values, metrics, readings, insights, graphs, or plots that are derived from at least a portion of the animal data. The sensors used herein initially provide an electronic signal. The computed asset is extracted or derived, at least in part, from the one or more electionic signals or its one or more derivatives. The computed asset describes or quantifies an interpetable property of the one or more targeted individuals. For example, electrocardiogram readings can be derived from analog front end signals (the electronic signal from the sensor), heart rate data (e.g., heart rate beats per minute) can be derived from electrocardiogram or PPG sensors, body temperature data can be derived from temperature sensors, perspiration data can be derived or extracted from perspiration sensors, glucose information can be derived from biological fluid sensors, DNA and RNA sequencing information can be derived from sensors that obtain genomic and genetic data, brain activity data can be derived from neurological sensors hydration data can be derived from in-mouth saliva or sweat analysis sensors, location data can be derived from GPS or RFID sensors, biomechanical data can he derived from optical or translation sensors, and breathing rate data can be derived from respiration sensors. ln a refinement, a computed asset can include one or more signals or readings from non-animal data sources as one or more inputs in its one or more computations, derivations incorporations, simulations, extractions, extrapolations, modifications, enhancements, creations, estimations, deductions, inferences, determinations, processes, communications, and the like. In another refinement, a computed asset is computed of a plurality of computed assets.

### Abbreviations

“BLE” means Bluetooth Low Energy.

“HALE” means high altitude long endurance.

“HAPS” means a high-altitude pseudo-satellite. It may also be referred to as an atmospheric satellite.

“RPAS” means a remotely piloted aerial system.

“UAV” means an unmanned aerial vehicle.

“VTOL” means vertical take-off and landing

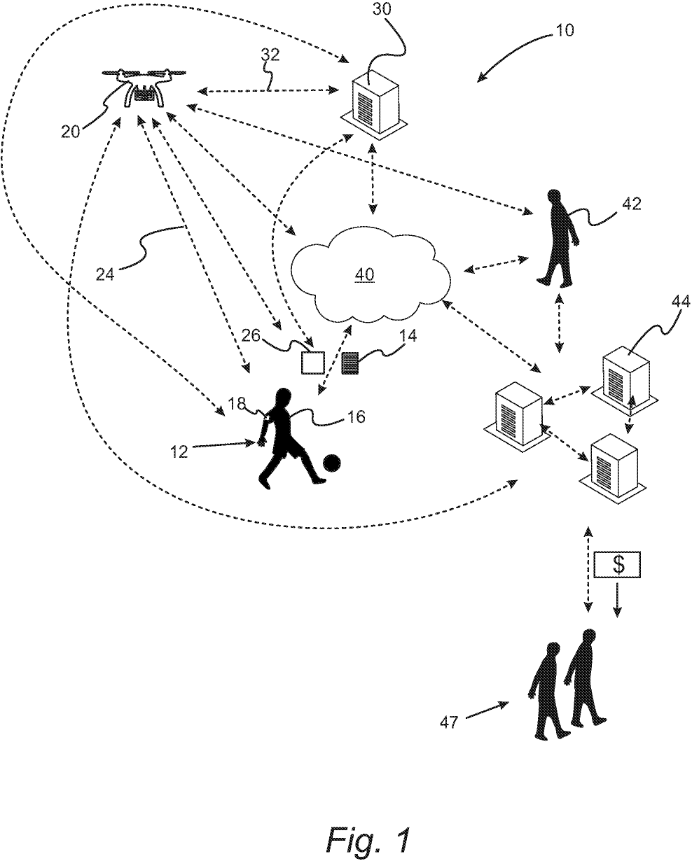

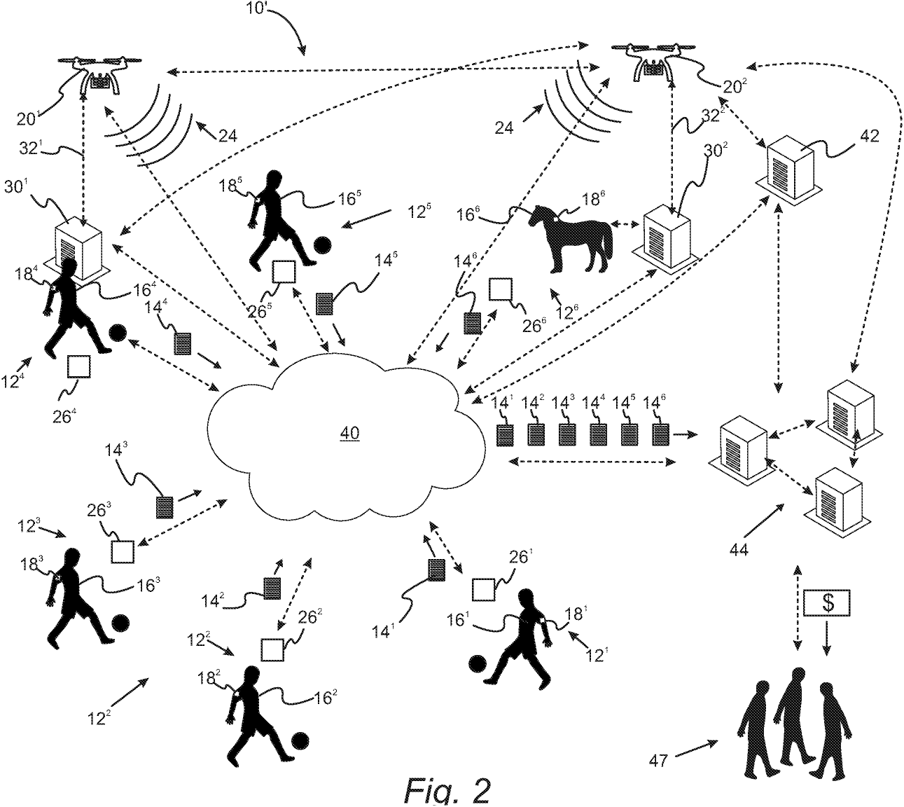

With reference to FIGS. 1 and 2, a schematic of a system for collecting and distributing animal data with one or more unmanned aerial vehicles is provided. UAV-based transmission system 10 and 10′ includes at least one source 12 of animal data 14 that can be transmitted electronically. Targeted individual 16 is the subject from which corresponding animal data 14 is collected. In this context, animal data refers to data related to a subject’s body obtained from sensors and, in particular, biosensors, as set forth below in more detail. Therefore, source 12 of animal data includes at least one sensor 18. In many useful applications, the subject is a human (e.g., an athlete, a soldier, a healthcare patient, a research subject, a participant in a fitness class), and the animal data is human data. Animal data 14 can be derived from a targeted individual 16, multiple targeted individuals 16, a targeted group of multiple targeted individuals 16, or multiple targeted groups of multiple targeted individuals 16. Animal data 14 can be obtained from a single sensor 18 on each targeted individual 16, or from multiple sensors 18 on each targeted individual 16. In some cases, a single sensor 18 can capture animal data 14 from multiple targeted individuals 16, a targeted group of multiple targeted individuals 16, or multiple targeted groups of multiple targeted individuals 16 (e.g., an optical-based camera sensor that can locate and measure distance run for a taigeted group of targeted individuals). Sensor 18 can provide a single type of animal data 14 or multiple types of animal data 14. In a variation sensor 18 can include multiple sensing elements to measure multiple parameters within a single sensor (e.g., heart rate and accelerometer data).

While FIG. 1 embodies a single targeted individual 16, FIG. 2 illustrates a scenario of FIG. 1 that includes a plurality of sources 12ⁱ, animal data 14ⁱ, targeted individuals 16ⁱ, and sensor 18ⁱ. In this context, “i” is merely a label to differentiate between different targeted individuals, sources, sensors, and animal data. It should be appreciated that the present embodiment is not limited by the number of targeted individuals 16, sources 12 of animal data 14, and/or sensors 18.

In a refinement, one or more sensors 18 include at least one biological sensor (biosensor). Biosensors collect biosignals, which in the context of the present embodiment are any signals as properties, in or derived from, animals that can be continually or intermittently measured, monitored, observed, calculated, computed, or interpreted, including both electrical and non-electrical signals, measurements, and artificially-generated information. A biosensor can gather biological data (e.g, including readings and signals) such as physiological data, biometric data, chemical data, biomechanical data, location data, genetic data, genomic data, or other biological data from one or more targeted individuals. For example, some biosensors may measure, or provide information that can be converted into or derived from, biological data such as eye tracking data (e.g., pupillary response, movement, EOG-related data) blood flow data and/or blood volume data (e.g., PPG data, pulse transit time, pulse arrival time), biological fluid data (e.g., analysis derived from blood urine, saliva, sweat, cerebrospinal fluid), body composition data, biochemical composition data, biochemical structure data, pulse data, oxygenation data (e.g., SpO2), core body temperature data, galvanic skin response data, skin temperature data, perspiration data (e.g., rate, composition), blood pressure data (e.g., systolic, diastolic., MAP), glucose data (e.g., fluid balance I/O), hydration data (e.g., fluid balance I/O), heart-based data (e.g., heart tate (HR), average HR, HR range, heart rate variability, HRV time domain, HRV frequency domain, autonomic tone, ECG-related data including PR, QRS, QT, RR intervals), neurological data and other neurological-related data (e.g FFG-related data) genetic-related data, genomic-related data, skeletal data, muscle data (e.g., EMG-related data including surface EMG, amplitude), respitatory data (e.g., respiratory rate, respiratory pattern, inspiration/expiration ratio, tidal volume, spirometry data), thoracic electrical bioimpedance data, or a combination thereof. Some biosensors may detect biological data such as biomechanical data which may include, for example, angular velocity, joint paths, kinetic or kinematic loads, gait description, step count, or position or accelerations in various directions from which a subject’s movements may be characterized. Some biosensor may gather biological data such as location and positional data (e.g., GPS, ultra-wideband RFID-based data such as speed, acceleration, or physical location; posture data), facial recognition data, audio data, kinesthetic data (e.g., physical pressure captuied from a sensor located at the bottom of a shoe), or auditory data related to the one or more targeted individuals. Some biological sensors are image or video-based and collect, provide, and/or analyze video or other visual data (e.g. still or moving images, including video, MRIs, computed tomography scans, ultrasounds, X-Rays) upon which biological data can be detected, measured, monitored, observed, extrapolated, calculated, or computed (e.g., biomechanical movements, location, a fracture based on an X-Ray, or stress or a disease based on video or image-based visual analysis of a subject). Some biosensors may derive information from biological fluids such as blood (e.g., venous, capillary), saliva, urine, sweat, and the like including triglyceride levels, red blood cell count, white blood cell count, adrenocorticotropic hormone levels, hematocrit levels, platelet count, ABO/Rh blood typing, blood urea nitrogen levels, calcium levels, carbon dioxide levels, chloride levels, creatinine levels, glucose levels, hemoglobin A1c levels, lactate levels, sodium levels, potassium levels, bilirubin levels, alkaline phosphatase (ALP) levels, alanine transaminase (ALT) levels, aspartate aminotransferase (AST) levels, albumin levels, total protein levels, prostate-specific antigen (PSA) levels, microalbuminuria levels, immunoglobulin A levels, folate levels, cortisol levels, amylase levels, lipase levels, gastrin levels, bicarbonate levels, iron levels, magnesium levels, uric acid levels, folic acid levels, vitamin B-12 levels, and the like. In addition to biological data related to one or more targeted individuals, some biosensors may measure non-biological data such as ambient tempetature data, humidity data, elevation data, barometric pressure data, and the like. In a refinement, one or more sensors provide biological data that include one or more calculations, computations, predictions, probabilities, possibilities, estimations, evaluations, inferences, determinations, deductions, observations or forecasts that are derived from, at least in part, biosensor data. In another refinement, the one or more biosensors are capable of providing two or more types of data, at least one of which is biological data (e.g., heart rate data and VO2 data, muscle activity data and accelerometer data, VO2 data and elevation data). In another refinement, the one or more sensors contain logic whereby the one or more readings provided is simulated data (e.g., artificial data generated via one or more simulations that provides information related to the probability or likelihood that an occunence will happen; an insight or computed asset that includes at least a portion of simulated data).

In a refinement, the at least one sensor 18 and/or one or more appendices of the at least one sensor can be affixed to, are in contact with, or send one or more electronic communications in relation to or derived from, the targeted individual including a targeted individual’s body, skin, eyeball, vital organ, muscle, hair, veins, biological fluid, blood vessels, tissue, or skeletal system embedded in a targeted individual, lodged or implanted in a targeted individual, ingested by a targeted individual, or integrated to include at least a portion of a targeted individual. For example, a saliva sensor affixed to a tooth, a set of teeth, or an apparatus that is in contact with one or more teeth, a sensor that extracts DNA information derived from a subject’s biological fluid or hair, a sensor (e.g., portable laboratory machine) that extracts biological fluid information from a subject’s blood sample, a sensor that is wearable (e.g., on a subject’s body), a sensor in a phone tracking a targeted individual’s location, a sensor affixed to or implanted in the targeted subject’s brain that may detect brain signals fiom neurons, a sensor that is ingested by a targeted individual to track one or more biological functions, a sensor attached to, or integrated with, a machine (e.g., robot) that shares at least one characteristic with an animal (e.g., a robotic arm with an ability to pet form one or more tasks similar to that of a human a robot with an ability to process information similar to that of a human), and the like. Advantageously, the machine itself may be comprised of one or more sensors and may be classified as both a sensor and a subject. In another refinement, the one or more sensors 18 are integrated into or as part of, affixed to, or embedded within, a textile, fabric, cloth, material, fixture, object, or apparatus that contacts or is in communication with a targeted individual either directly or via one or more intermediaries or interstitial items. Examples include a sensor attached to the skin via an adhesive, a sensor integrated into a watch or headset, a sensor integrated or embedded into a shirt or jersey (e.g., of a pro sports team), a sensor integrated into a steering wheel, a sensor integrated into a video game controller, a sensor integrated into a basketball that is in contact with the subject’s hands, a sensor integrated into a hockey stick or a hockey puck that is in intermittent contact with an intermediary being held by the subject (e.g., hockey stick), a sensor integrated or embedded into the one or more handles or grips of a fitness machine (e.g., treadmill, bicycle, bench press), a sensor that is integrated within a robot (e.g., robotic arm) that is being controlled by the targeted individual, a sensor integrated or embedded into a shoe that may contact the targeted individual through the intermediary sock and adhesive tape wrapped around the targeted individual’s ankle, and the like. In another refinement, one or more sensors may be interwoven into, embedded into, integrated with, or affixed to, a flooring or ground (e.g., artificial turf, grass, basketball floor, soccer field, a manufacturing assembly-line floor), a seat/chair, helmet, a bed, an object that is in contact with the subject either directly or via one or more intermediaries (e.g., a subject that is in contact with a sensor in a seat via a clothing interstitial), and the like. In another refinement, the sensor and/or its one or more appendices may be in contact with one or more particles or objects derived of the subject’s body (e.g., tissue from an organ, hair from the subject) from which the one or more sensors derive, or provide information that can be converted into, biological data. In yet another refinement, one or more sensors may be optically-based (e.g., camera-based) and provide an output from which biological data can be detected, measured, monitored, observed, extracted, extrapolated, inferred, deducted, estimated, determined, calculated, or computed. In yet another refinement, one or more sensors may be light-based and use infrared technology (e.g., temperature sensor or heat sensor) or other light-based technology to calculate the temperature of a targeted individual or the relative heat of different parts of a targeted individual. In yet another refinement, one or more sensor 18 include a transmitter, receiver, or transceiver.

Specific examples of biosensors include, but are not limited to, the Mc10 BioStamp nPoint (ECG + sEMG + XYZ coordinates), Vivalnk Vital Scout (ECG); Humon Hex (muscle oxygen); Apple Watch (heart rate); Polar H10 chest strap (heart rate and HRV); 23andMe testing technologies (DNA/genetic testing); Nebula Genomics testing technologies (genomic testing); NEC NeoFace Watch (facial recognition); Sonitus technologies MolarMic (auditory); SennoFit Insole (gait analysis); Omron HeartGuide Wearable Blood Pressure Monitor, model: BP-8000M (blood pressure); Boston Dynamics Spot robot (visual data); Abbott freestyle Libre (glucose); Health Care Originals ADAMM (respiration rate); Epicore Biosystems (hydration/sweat analysis); Kenzen Echo Smart Patch (hydration/sweat analysis); IsoLynx Athlete Tracking Tags and Wireless Smart Nodes (RFID-based location tracking); Catapult OptimEye S5 (GPS location tracking); SMRT Mouth (biometric mouth guard); StrikeTec (biomechanical movement sensors for fight sports); Scanalytics (smart floor sensors); Tesla Model X (cognitive data); Wellue O2 Ring (oxygenation data); Genalyte Maverick Diagnostic System (biological fluid analysis); Microlife NC 150 BT (body temperature); and Lockheed Martin FORTIS industrial exoskeleton products (biomechanical movements).

Still referring to FIGS. 1 and 2, UAV-based transmission systems 10 and 10′ include unmanned aerial vehicle 20 that electronically communicates directly or indirectly with one or more sensors 18 to gather animal data 14 from source 12. Typically, electronic communication occurs by receiving one or more signals from one or more sensors 18 that gather information from one or more targeted individuals 16, which can occur wirelessly via wireless links 24. The one or more unmanned aerial vehicles 20 can he used to collect, process (e.g., transform), and distribute information related to one or more targeted individuals 16 to one or more endpoints (e.g., servers, computing devices, and the like that are operable to access or receive information from the one or more unmanned aerial vehicles either directly or indirectly). In a refinement, unmanned aerial vehicle 20 is operable to send at least a portion of the animal data 14 to another computing device. In a variation, a computing device is at least one of; a home station 30, an intermediary server 44, a third party computing device 42, a cloud server 40, another unmanned aerial vehicle 20, or other computing devices (e.g., computing device 26). Unmanned aerial vehicle 20 can be a single unmanned aerial vehicle 20 or multiple unmanned aerial vehicles 20^{j} that operate independently or together within a network or plurality of networks. In this context, “j” is an integer label differentiating the multiple unmanned aerial vehicles in FIG. 2. It should be appreciated that the present invention is not limited by the number of unmanned aerial vehicles utilized. In this regard, the one or more unmanned aerial vehicles 20 can operate as one or more interrelated or interacting components that coordinate one or more actions to achieve one or more common goals or produce one or more desired outcomes. Typically, unmanned aerial vehicle 20 contains a transmission subsystem that includes a transmitter and a receiver, or a combination thereof (e.g., transceiver). The transmission subsystem can include one or more receivers, transmitters and/or transceivers having a single antenna or multiple antennas, which may be configured as part of a mesh network and/or utilized as part of an antenna array. The transmission subsystem and/or its one or more components may be housed within or as part of the one or more unmanned aerial vehicles or may be external to the one or more unmanned aerial vehicles (e.g., a dongle connected to the unmanned aerial vehicle which is comprised of one or more hardware and/or software components that facilitate wireless communication and is part of the transmission subsystem). In a refinement, the one or more unmanned aerial vehicles include one or more computing devices operable to take one or more actions (e.g., processing steps upon the collected animal data, receive, create, and send commands; and the like).

The one or more unmanned aerial vehicles 20 can be operable to communicate electronically with the one or more sensors 18 from the one or more targeted individuals 16 using one or more wireless methods of communication via communication links 24. In this regard UAV-based transmission system 10 and 10′ can utilize any number of communication protocols and conventional wireless networks to communicate with one or more sensors 18 including, but not limited to, Bluetooth Low Energy (BLF), ZigBee, cellular networks, LoRa, ultra-wideband, Ant+, WiFi, and the like. The present invention is not limited to any type of technologies or communication links (e.g., radio signals) the one or more sensors 18, unmanned aerial vehicles 20, and/or any other computing device utilized to transmit and/or receive signals. In a refinement, unmanned aerial vehicle 20 is operable to electronically communicate with at least one sensor 18 from one or more targeted individuals using one or more wireless communication protocols. Advantageously, the transmission subsystem enables the one or more sensors 18 to transmit data wirelessly for real-time or near real-time communication. In this context, near real-time means that the transmission is not purposely delayed except for necessary processing by the sensor and any other computing device. In a variation, one or more unmanned aerial vehicles 20 can communicate with the one or more sensors 18 from the one or more targeted individuals 16 using one or more communication protocols simultaneously. For example, targeted individual(s) 16 may be wearing two separate sensor that transmit information utilizing different communication protocols (e.g. , BLE and Ant+). In this scenario, UAV 20 can be operable to communicate with both sensors simultaneously by utilizing the primary method of communication found on each of the one or more sensors. In another example, UAV-based cellular networks may be utilized to communicate with a plurality of sensors from a group of targeted individuals. In a refinement, multiple unmanned aerial vehicles 20 are operable to receive data from the same one or more sensors 18. In another refinement, one or more unmanned aerial vehicles 20 gather information from the one or more sensors 18 via communication with a server such as cloud 40. The cloud 40 server can be the internet, a public cloud, private cloud, or hybrid cloud. In another refinement, the one or more unmanned aerial vehicles 20 communicate with the one or more sensors 18, computing devices 26, and/or home stations 30 via cloud 40. In another refinement, the one or more UAVs 20 can functionally serve, at least in part as cloud 40. In another refinement, unmanned aerial vehicle 20 is operable to electronically communicate with two or more sources 12 of animal data 14 simultaneously (e.g., multiple sensors 18, or a sensor 18 and a cloud server 40 that has animal data). In another refinement, two or more unmanned aerial vehicles 20 are operable to electronically communicate with the same source 14 of animal data 12.

Still referring to FIGS. 1 and 2, source 12 of animal data 14 may include computing device 26 which mediates the sending of animal data 14 to one or more unmanned aerial vehicles 20 (e.g., it collects the animal data and transmits it to one or more unmanned aerial vehicles 20). In some cases, computing device 26 is local to the targeted individual or group of targeted individuals. For example, computing device 26 can be a smartphone, smartwatch, tablet, or a computer earned by or proximate to targeted individual 16. However, computing device 26 can be any computing device, including devices that do not have displays (e.g., a display-less transceiver with one or more antennas). In another variation, computing device 26 may also be part of unmanned aerial vehicle 20. Computing device 26 can include a transceiver and can be operable to electronically communicate with the one or more sensors 18 on a targeted individual or across multiple targeted individuals. Advantageously, computing device 26 may be operable to act as an intermediary computing device and collect the one or more data streams from one or more sensors 18 on one or more targeted subjects 16 prior to the data being sent wirelessly to the one or more unmanned aerial vehicles 20. Computing device 26 can be operable to communicate with each sensor using a communication protocol of that particular sensor and aggregating at least a portion of the sensor data so that the one or more unmanned aerial vehicles 20 can communicate with a single source (e.g., computing device 26) to receive the one or more data streams. In this regard, computing device 26 can act as a data collection hub and communicate with the one or more unmanned aerial vehicles 20 via any number of communication protocols or wireless communication links (e.g., radio signals) utilized by the UAV including, but not limited to, BLE, cellular networks, LoRa, ultra-wideband, Ant+, WiFi, ZigBee, and the like. The present invention is not limited to any types of technologies or communication links the one or more unmanned aerial vehicles 20 and the one or more computing devices 26 utilize to transmit and/or receive signals. In a refinement, computing device 26 is configured to optimize the one or more UAVs (e.g., minimize the transmission overhead) by containing logic that takes one or more actions (e.g., processing steps) upon the data collected from one or more sensors 18 prior to sending data to the UAV. For example computing device 26 may collect normalize, timestamp, aggregate, store, manipulate, denoise, enhance, organize, analyze, summarize, replicate, synthesize, anonymize, or synchronize the data prior to being sent to the one or more unmanned aerial vehicles 20. Advantageously, one or more functions performed by computing device 26 can reduce communication-related constraints (e.g., power bandwidth) of the one or more UAVs. For example, by enabling computing device 26 to collect data signals from one or more sensors, aggregate data signals from multiple sensors, or summarize data sets, computing device 26 can reduce the amount of transmission-related energy required by the one or more UAVs (e.g., instead of communicating with multiple sensors, the UAV only needs to communicate with computing device 26; computing device 26 may be operable to collect more data points per second from the one or more sensors and reduce the amount of data being sent to the one or more UAVs per second), as well as reduce the number of actions taken by the one or more UAVs (e.g., once received, the one or more UAVs may take less processing steps on the data, such as less computations or analysis). In a refinement, computing device 26 can be UAV 20. In a variation, computing device 26 may operate as an on-ground computing device (e.g., base station) with one or more transceivers equipped with one or more antennas within a network or plurality of networks. In another refinement, computing device 26 tracks one or more types of biological data (e.g., positional or location data). In another refinement, computing device 26 is an on or in-body transceiver affixed to integrated with, or in contact with, a targeted subject’s skin, hair, vital organ, muscle, skeletal system, eyeball, clothing, object, or other apparatus on a subject. In another refinement, computing device 26 can communicate with multiple sensors 18 simultaneously, utilizing either the same communications protocol for each sensor 18 or different communication protocols with the multiple sensors 18. In another refinement, computing device 26 can communicate (e.g., receive data, send commands, send data) with one or more sensors 18, UAVs 20, home stations 30, clouds 40, or a combination thereof, simultaneously. In another refinement, a plurality of computing devices 26 can communicate (e.g., receive data, send commands, send data) with the same one or more sensors 18, UAVs 20, home station 30, clouds 40, or a combination thereof, simultaneously. In another refinement, a single UAV 20 can communicate with a plurality of computing devices 26^{k}. In this context, “k” is an integer label differentiating the multiple computing devices 26 in FIG. 2. In another refinement, a plurality of UAVs 20 can communicate with the same computing device 26. In another refinement, a plurality of UAVs 20 can communicate with a plurality of computing devices 26. In another refinement, computing device 26 can communicate with the one or mote UAVs 20 or sensors 18 via cloud 40 In yet another refinement, computing device 26 is operable to receive data from the one or more UAVs 20.

Still referring to FIGS. 1 and 2, to initially establish electronic communication links between the one or more sensors 18 and the one or more UAVs 20, home station 30 can be utilized. A function of home station 30 is to manage (e.g., establish, monitor, troubleshoot) the one or more communications between the one or more sensors 18, the one or more UAVs 20, and any one or more other computing devices or clouds that are part of a UAV network or plurality of UAV networks via a control application. Advantageously, home station 30 can be comprised of a plurality of home stations 30^{m}, with each home station operable to execute the same one or more actions or different one or more actions within a network or plurality of networks (e.g., in the case of having different functionality, one home station may establish communication and set up the sensors, while another home station monitors the one or more UAVs) In this context, “m” is an integer label differentiating the multiple home stations in FIG. 2 One or more unmanned aerial vehicles 20 communicate with home station 30 via one or more direct communication links 32 (e.g. radio signals) or via cloud 40 (e.g., private cloud, public cloud, hybrid cloud). Home station 30 can be a computing device such as a server, laptop, mobile device (e.g., smartphone, smart watch, smart glasses), tablet, a programmable logic array (e.g., a field-programmable logic array), or any other computing device capable of operating the functions of home station 30 described herein. In a variation, a user selects a sensor and opens a control application for the sensor on a home station 30. Home station 30 can be programmed to enable a user to initiate communication with a single sensor 18 or multiple sensors 18 for a single targeted individual 16, or a single sensor 18 or multiple sensors 18 for multiple targeted individuals 16. In this regard home station 30 can be operable to communicate with one or more sensors 18 (e.g., send commands, receive data), either directly (e.g., initial direct communication links between one or more sensors 18 and home station 30) or indirectly. Indirect communication can include home station 30, establishing communication with one or more sensors 18 via UAV 20, computing device 26, or cloud 40. Initiating electronic communication can also include establishing communication (e.g., pairing) with one or more sensors and another computing device (e.g., home station 30, computing device 26, unmanned aerial vehicle 20). Typically, the one or more sensors 18 have been previously integrated with the control application operating on home station 30 prior to communicating with UAV 20. Advantageously, a single home station can communicate, either directly or indirectly with a single UAV 20 or a plurality of UAVs 20, either independently or as part of a network. Home station 30 can also act as an administrator for a network of UAVs 20 or a plurality of networks. In this role, home station 30 can be operable to create, configure, change, control, and/or modify one or more networks. This can include an ability for home station 30 to send one more commands to the one or more UAVs 20 (e.g., turn on/off, change position, change location, change multi-UAV formation), send one or more commands the one or more sensors 18, and the like. Control of the one or more UAVs can include manual control (e.g., conventional joystick to control movement, verbal commands to control movement) or automated control (e.g., programmed to take one or more actions). In a refinement, one or more home stations 30 can be utilized to send one or more commands to computing device 26. Advantageously, the one or more commands can include control commands (e.g., an ability to control one or more UAVs 20, sensors 18, or computing devices 26). In a variation, one or more home stations can be in communication with different UAVs, which may be part of a network, but provide data to the same one or more endpoints (e.g., computing devices). For example, multiple UAVs may be communication with different home stations but communicate with the same cloud 40. In a refinement, home station 30 utilizes one or more artificial intelligence techniques to control (e.g., change), at least in part, one or more functions of the one or more UAVs 20, sensors 18, computing devices 26, home stations 30, or a combination thereof.

In a refinement, home station 30 is operable to establish one or more communication links between one or more sensors 18 and one or more UAVs 20. For example, home station 30 can be programmed to initiate communication with sensor 18 via UAV 20. In a variation, the one or more communication links may be part of a network or plurality of networks established between one or more sensors 18 and one or more UAVs 20. In another refinement, home station 30 is operable to establish one or more communication links between one or more sensors 18, home stations 30, and UAVs 20 For example, home station 30 can initiate communication with sensor 18, initiate communication with LAV 20, and then provide one or more commands to initiate communication between sensor 18 and LAV 20. In a variation, the one or more communication links may be part of a network or plurality of networks established between one or more sensors 18, home stations 30, and UAVs 20. In another refinement, home station 30 is operable to establish one or mote communication links between one or more sensors 18, computing devices 26, and UAVs 20, For example, home station 30 can initiate communication with sensor 18 and UAV 20 via communication with computing device 26. which in turn can initiate communication with sensor 18 and UAV 20, and then between sensor 18 and UAV 20. In a variation, computing device 26 may act as a data collection hub for the one or more sensors 18 and communicate with one or more UAVs 20 on behalf of the one or more sensors 18. In another variation, the one or more communication links may be part of a network or plurality of networks established between one or more sensors 18 computing devices 26, and UAVs 20. In another refinement, home station 30 is operable to establish one or more communication links between one or more sensors 18, home stations 30, computing devices 26, and UAVs 20. For example, home station 30 can initiate communication with sensor 18 via communication with computing device 26 (which in turn is programmed to establish communication with sensor 18). Home station 30 can also initiate communication with UAV 20. Upon establishing communication with sensor 18 (via computing device 26) and UAV 20, home station 30 can provide one or more commands to initiate communication between sensor 18 and UAV 20. In a variation, home station 30 may initiate communication between UAV 20 and computing device 26 in the event computing device 26 acts as a data collection hub for the one or more sensors 18 operable to communicate with the one or more UAVs. In another variation, the one or more communication links may be part of a network or plurality of networks established betw een one or more sensors 18, computing devices 26, home stations 30, and UAVs 20. In another refinement, home station 30 is operable to establish one or more communication links between one or more sensors 18, home stations 30, clouds 40 and UAVs 20. For example, home station 30 can initiate communuation with sensor 18 and UAV 20 via communication with one or more clouds 40 (which may be associated with home station 30, one or more sensors 18, one or more UAVs 20, or a combination thereof as part of a network). Upon establishing communication with sensor 18 and UAV 20, home station 30 can provide one or more commands (e.g., direct communication link, communication link via cloud 40) to initiate communication between sensor 18 and UAV 20 In a variation, the one or more communication links may be part of a network or plurality of networks established between one or more sensors 18, clouds 40. home stations 30, and UAVs 20. In another refinement, home station 30 is operable to establish one or more communication links between one or more sensors 18 home stations 30. computing devices 26, clouds 40, and UAVs 20. For example, home station 30 can initiate communication with sensor 18 via communication with computing device 26 (which in turn is programmed to initiate communication with sensor 18) Home station 30 can also initiate communication with UAV 20. One or more of the communication links may be established via cloud 40. Upon establishing communication with sensor 18 and UAV 20, home station 30 can provide one or more commands to initiate communication between sensor 18 and UAV 20. In a variation, the one or more communication links may be part of a network or pluarlity of networks established between one or more sensors 18, computing devices 26, clouds 40, home stations 30, and UAV 20. In another refinement computing device 26 is operable to take one or more actions on behalf of home station 30 (e.g., communicate one or more functions, send commands to the one or more sensors, send commands to the one or more UAVs). In another refinement, computing device 26 can operate, at least in part, as home station 30.

In another refinement, a plurality of home stations 30 can be utilized to control a single UAV or a plurality of UAVs which may be part of a network or plurality of networks. Utilizing a plurality of home stations that operate together within a network or plurality of networks can enable each home station to sharehome sensor duties (or have separate, defined duties within a network), share or define control the one or more UAVs (e.g., provide commands), coordinate communications with one or more computing devices 26, and the like. In another refinement a single home station 30 may operate as the parent home station to one or more other home stations 30 within a network or plurality of networks. In another refinement, the one or more home stations operate independently of each other and communicate with different UAVs that are also operating independently of each other, but provide one or more commands to send the collected sensor data to the same end point (e.g., computing system operating on a computing device). For example, multiple UAVs may be communication with different home stations but communicate with the same cloud 40. In another refinement a single UAV 20 can communicate with a plurality of home stations 30. In another refinement, a plurality of UAVs 20 can communicate with the same home station 30. In another refinement, home station 30 can communicate (e g receive data, send commands, send data) with one or more sensors 18, computing devices 26, UAVs 20, clouds 40, or a combination thereof, simultaneously. In yet another refinement, a plurality of home stations can communicate (e.g., receive data, send commands, send data) with the same one or more sensors 18, computing devices 26, UAVs 20, clouds 40, or a combination thereof, simultaneously.

Still referring to FIGS. 1 and 2 upon receiving animal data 14 (e.g., from the one or more sensors 18 either directly or indirectly) the one or more unmanned aerial vehicles 20 take one or more actions (e.g., processing steps) with at least a portion of the received animal data. The one or more actions can include attaching metadata to the collected animal data. In a refinement, the unmanned aerial vehicle attaches metadata to the animal data. Characteristically, metadata includes any set of data that describes and provides information about other data, including data that provides context for other data (e.g., the one or more activities a targeted individual is engaged in while the animal data is collected conditions in which the data was collected, contextual information). Metadata can include one or more characteristics of the animal data (e.g., data type, timestamps, location, origination), origination of the animal data, sensor-related data (including the type of sensor, operating parameters, mode), and the like. Other information including one or more attributes related to the one or more targeted individuals from which the animal data originates, one or more attributes related to the sensor, one or more attributes related to the data, and/or one or more attributes related to the UAV, can also be included as part of the metadata or associated with the animal data by the one or more unmanned aenal vehicles 20 after the animal data is collected (e.g., height, age, weight, data quality assessments, UAV location, UAV model number, and the like). In a refinement, the metadata includes one or more characteristics related to the at least one targeted individual, the at least one sensor the unmanned aenal vehicle, the animal data, or combination thereof. In a variation, one or more other computing devices can also attach metadata upon receiving the data from unmanned aerial vehicle 20 (e.g. home station 30, third-party system 42, intermediary server 44, computing device 26) or upon receiving data from the sensor or computing device 26.

In a refinement, the one or more actions are selected from the group consisting of normalizing the animal data, associating a timestamp with the animal data, aggregating the animal data, applying a tag to the animal data, storing the animal data, manipulating the animal data, processing the data, denoising the animal data enhancing the animal data organizing the animal data, analyzing the animal data, anonymizing the animal data, visualizing the animal data, synthesizing the animal data, summarizing the animal data, synchronizing the animal data, replicating the animal data, displaying the animal data, distributing the animal data, productizing the animal data, performing bookkeeping on the animal data, or combinations thereof In another refinement, the one or more actions includes at least one coordinated action with another computing device upon the same set of received animal data.

The one or more actions taken by the one or more unmanned aerial vehicles 20 can also include taking one or more processing steps to transform sensor data. For the purposes of this invention, each step in a process that takes one or more actions upon the data can be considered a transformation. In this context, one or more processing steps can include one or more calculations computations, derivations, incorporations, simulations, extractions, extrapolations, modifications, enhancements creations, estimations, deductions, inferences, determinations, and the like. In a variation, sensor data may be transformed into one or more computed assets or insights. For example, in the context of calculating a computed asset such as a heart rate, sensor 18 may be operable to measure electric signals from targeted individual 16, transforming (e.g., converting) analog measurements to digital readings, and transmitting the digital readings. Unmanned aerial vehicle 20 can receive the digital readings and transform the digital readings into one or more heart rate values via one or more calculations based on overlapping segments of the digital readings by (i) identifying R-peaks within the overlapping segments of the ECG measurements, (ii) calculating a number of sample values based on times between adjacent R-peaks, (iii) discarding samples that are influenced by false peak detection or missed peak detection, and (iv) calculating an average, which may be weighted, of remaining sample values.

In a refinement of one or more transformations related to calculating a heart rate that can occur utilizing one or more unmanned aerial vehicles, the at least one biological sensor 18 may be operable to measure electric signals in a targeted subject’s body, convert one or more analog measurements to one or more digital readings and transmit the one or more digital readings. In this case, the unmanned aerial vehicle can be configured to receive the one or more digital readings and calculate heart rate based on one or more overlapping segments of the one or more digital readings by identifying R-peaks within the one or more overlapping segments of the ECG measurements, calculating one or more sample values based on times between adjacent R-peaks, discarding one or more samples that are influenced by false peak detection or missed peak detection, and calculating one or more averages of remaining sample values. The unmanned aerial vehicle can be operable to provide the one or more averages of the remaining sample values to one or more computing devices (e.g., another UAV 20, home station 30, third party 42, intermediary server 44, computing device 26, cloud 40). In a variation, the one or more averages of the remaining sample values may be sent to another one or more UAVs 20 which in turn are sent to one or more computing devices.

In another refinement of the one or more transformations related to calculating a heart rate that can occur utilizing one or more unmanned aerial vehicles, the at least one biological sensor 18 may he adapted tor fixation to a targeted subject’s skin and configured to measure electric signals in the skin, convert analog measurements to digital readings, and transmit the digital readings. In this case, the unmanned aerial vehicle receives the digital readings and utilizes logic incorporated as part of the unmanned aerial vehicle (e.g., the logic contained within the UAV or within the cloud associated and in communication with the UAV) to calculate the one or more heart rate values based on one or more overlapping segments of the digital readings by (i) identifying R-peaks within the one or more overlapping segments of the ECG measurements, (ii) calculating a number of sample values based on times between adjacent R-peaks, (iii) selecting samples within a first threshold of a previous heart rate value, and (iv) setting a current heart rate value to an average of the selected samples which may be weighted. Each simple value may be proportional to a reciprocal of a time between adjacent R-peaks. The logic incorporated as part of the unmanned aerial vehicle may select samples within a second threshold ot the previous heart rate value in response to a standard deviation of differences between consecutive samples being greater than a thud threshold. The logic contained on the unmanned aerial vehicle may set the current heart rate value equal to the previous heart rate value in response to the number ot samples being less than a fourth threshold or in response to no samples being selected. The unmanned aerial vehicle can then communicate the one or more current heart rate values to one or more endpoints (e.g., computing devices). The logic and system onboard the UAV may operate in real-time or near real-time wherein the unmanned aerial vehicle makes available each current heart rate value before a respective succeeding heart rate value is calculated and the unmanned aerial vehicle calculates each current heart rate value before the at least one sensor 18 completes measuring at least a portion of or all of the readings used to calculate the succeeding heart rate value. The logic contained on the unmanned aerial vehicle may compute an initial heart rate value by receiving a preliminary segment of the digital readings longei than the overlapping segments, identifying R-peaks within the preliminary segment, calculating sample values based on times between adjacent R-peaks, and calculating an average of the samples, which may be weighted.

In yet another refinement of one or more transformations related to calculating a heart rate that can occur utilizing one or more unmanned aerial vehicles, the at least one biological sensor 18 may be configured to measure one or more electric signals to a targeted subject’s body, transform (e g., convert) analog measurements to one or more digital readings, and transmit the digital readings. In this case, the unmanned aerial vehicle can be configured to receive the one or more digital readings from the one or more sensors 18 and utilize onboard logic to transform (e.g., calculate) one or more heart rate values based on one or more overlapping segments of the one or more digital readings by identifying R-peaks within the one or more overlapping segments of the ECG measurements, calculating one or more sample values based on times between adjacent R-peaks, selecting one or more samples within a first threshold of a previous heart rate value, and setting a current heart rate value to an average of selected samples. In a variation, upon receiving the one or more digital readings, unmanned aerial vehicle 20 sends the data to cloud 40. Data can then be transformed in cloud 40 and accessed by another one or more computing devices via cloud 40, or the data is sent to the same UAV 20 or another one or more UAVs 20 for distribution to one or more computing devices (e.g., home station 30, intermediary server 44 third party 42, other one or more UAVs 20 computing device 26). In another variation, upon receiving the one or more digital readings unmanned aerial vehicle 20 sends the data to home station 30, intermediary server 44, third party 42, cloud 40, or computing device 26 for transformation. In another variation, upon receiving the one or more digital readings, unmanned aerial vehicle 20 sends the data to another one or more UAVs 20 for transformation. In this example, the one or more UAVs may execute a senes or coordinated processing steps on the data to transform the data (e.g., into a computed asset). Each of the one or more processing steps may occur on different UAVs. In yet another variation upon receiving the one or more digital readings unmanned aerial vehicle 20 takes one or more actions prior to sending data (e.g., direct, indirect via cloud 40), to another one or more UAVs 20 for further transformation.

In still another refinement of one or more transformations related to calculating a heart rate that can occur utilizing one or more unmanned aerial vehicles, one or more readings are received by unmanned aerial vehicle 20 from the at least one biological sensor 18, with the unmanned aerial vehicle 20 operable to process the one or more readings. For example, a first segment of readings is received by the unmanned aeual vehicle from the one or more sensors. R-peaks within the first segment of ECG measurements are then identified by the unmanned aerial vehicle. Then, a first plurality of sample values is calculated by the unmanned aerial vehicle based on times between adjacent R-peaks. For example, a constant may be divided by times between adjacent R-peaks. A first subset of the first plurality ot sample values are selected including only sample values within a first threshold of a previous heart rate value. Then, a first updated heart rate value is calculated by the unmanned aerial vehicle based on an average of the first subset of sample values. The first heart rate value may then be sent by the unmanned aerial vehicle to one or more computing devices for display (e.g., third patty 42, computing device 26, home station 30). In later iterations, a second segment of the digital readings may be received by the unmanned aerial vehicle 20 from the one or more sensors 18. A third segment of digital readings may he formed by appending the second segment to the first segment R-peaks within the third segment may then be identified. A second plurality of sample values may be calculated based on times between adjacent R-peaks. Then, a plurality of differences between consecutive samples may be calculated. In response to a standard deviation of the differences exceeding a second threshold, a second subset of the second plurality of sample values may be selected, including only sample values within a third threshold of the first updated heart rate value. A second updated heart rate value mav then be calculated by the unmanned aerial vehicle based on an average of the second subset of sample values, which may be weighted. The second heart rate value may then be sent by the unmanned aerial vehicle to one or more computing devices for display. An initial heart rate value may be calculated based on a preliminary segment of the digital readings. In a refinement a plurality of unmanned aerial vehicles 20 may operate within a network or plurality of networks to take one or more actions upon the same data, with each UAV having a specified role in the transformation of the data.

In still another refinement of one or more transformations related to a biological measurement (e.g., heart rate value) that can occur utilizing one or more unmanned aerial vehicles, transformation via the one or more UAVs occurs when addressing issues related to signal quality. In cases where the raw data from the one or more sensors 18 has an extremely low signal-to-noise ratio, additional pre-filter logic that is associated with the one or more UAVs (e.g., onboard the UAV, in the cloud in communication with the UAV) may be applied to transform the data prior to calculating a biological measurement. In another method the pre filter process detects any outlier values and replaces the one or more outlier values, using a look-ahead approach, with values that align in the time series of generated values and fit within a preestablished threshold range. These generated values that fit within a preestablished threshold/range can be passed along through the system for its computation of the one or more biological measurements.

In yet another refinement of one or more transformations related to a biological measurement that can occur utilizing one or more unmanned aerial vehicles, transformation occurs via the one or more UAVs when detecting and replacing one or more outlier values generated from one or more biological sensors 18. Unmanned aerial vehicle 20 can be operable to receive one or more values generated directly or indirectly by one or more biological sensors. One or more statistical tests can be applied via logic utilized by unmanned aerial vehicle 20 (e.g., onboard the UAV, in the cloud in communication with the UAV) to deter mine an acceptable upper and/or lower bound for each value. A backward filling method can be used to replace the one or more outlier values with a next available value that falls within an acceptable range established in a current window of samples. Additional details related to a system for measuring a heart rate and other biological data are disclosed in U.S. Pat. Application No. 16 246,923 filed Jan. 14, 2019 and U.S. Pat. No. PCT/US20/13461 filed Jan. 14, 2020; the entire disclosures of which are hereby incorporated by reference. The present invention is not limited to the methods or systems used to transform animal data, including its one or more derivatives, nor is the present invention limited to the type of data being transformed. In another refinement, the act of transforming data includes one or more processing steps selected from the group consisting of normalizing the animal data, associating a timestamp with the animal data aggregating the animal data, applying a tag to the animal data, adding metadata to the animal data, storing the animal data, manipulating the animal data, denoising the animal data, enhancing the animal data organizing the animal data, analyzing the animal data, anonymizing the animal data, processing the animal data visualizing the animal data synthesizing the animal data summarizing the animal data, synchronizing the animal data, replicating the animal data, displaying the animal data distributing the animal data, productizing the animal data performing bookkeeping on the animal data, and combinations thereof. In still another refinement, one or more transformations occur utilizing one or more signals or readings (e.g. inputs) from non-animal data. In yet another refinement, one or more intermediary servers 44, third-party systems 42, computing devices 26 clouds 40, and/or home stations 30 are operable to transform sensor data.

Still refetencing to FIGS. 1 and 2, collected animal data is provided (e.g., transmitted, accessed, sent, made available) by the one or more unmanned aerial vehicles 20 to one or more third party computing devices 42, intermediary servers 44, computing devices 26, home stations 30, another one or more UAVs 20, or a combination thereof via direct communication links or cloud 40. Characteristically, the one or more UAVs may be operable to provide any collected and selected data (e.g., animal data, computed assets any derivatives and the like) to any number of computing devices in real-time or near real-time, while enabling any data not selected to be stored on the one or more UAVs and/or with any associated cloud 40 for access at a later time. It should be appreciated that intermediary server 44 is a computing device that can receive the animal data with or without the metadata and attributes attached thereto. Moreover, intermediary server 44 can implement the same operations described herein as home station 30 regarding taking one or more actions on the data (e.g., transforming the data), as well as similar operations as the one or more UAVs (e.g., provide data to one or more third parties either via direct communication links or indirectly via a mechanism such as making the data available for access via cloud 40). In a refinement, intermediary server 44 operates as cloud 40. In another refinement, cloud 40 operates as intermediary server 44. Third-party 42 is any computing device (e.g., which includes systems operating on that computing device) that can receive data provided by the one or more UAVs either directly or indirectly. One or more third-party computing devices 42 can include sports media systems (e.g., for displaying the collected data), insurance provider systems, telehealth systems, sports wagering systems, analytics systems, health and wellness monitoring systems (e.g., including systems to monitor viral infections), fitness systems, military systems hospital systems emergency response systems and the like. It can also include systems located on the one or more targeted individuals (e.g, another wearable with a display such as a smartwatch or VR headset) or other individuals interested in accessing the targeted individuals data (e.g., military commander interested in accessing the animal data from one or more targeted individual soldiers on their computing device such as then mobile command system). In a refinement, one or more actions (e.g., processing steps) may be taken on the same sensor data by two or more computing devices to transform the sensor data. For example, UAV 20 may synchronize the animal data while intermediary server 44 analyzes the data. In another example, UAV 20 may apply one or more tags to the received animal data and send the animal data to another UAV (e.g., within the same network) to analyze the received data.

Electronic communication from one or more unmanned aerial vehicles 20 to one or more home stations 30, third-party systems 42, intermediary servers 44, computing device 26, another one or more UAVs, or a combination thereof, can occur either directly (e.g., direct communication links) or indirectly (e.g., via cloud 40). For example, one or more unmanned aerial vehicles 20 can communicate with home station 30 via communication link 32. Alternatively, the one or more unmanned aerial vehicles 20 can communicate with home station 30 via cloud 40. Advantageously, UAV-based transmission system 10 and 10′ can utilize any number of communication protocols and conventional wireless networks to communicate with one or more computing devices (e.g., home station 30, third-party system 42, intermediary server 44, cloud 40, computing device 26). In a refinement, a single unmanned aerial vehicle 20 can communicate with one or more of third-parties 42, intermediary servers 44, home stations 30, clouds 40, computing devices 26, other unmanned aerial vehicles 20, or a combination thereof. In another refinement, a plurality of unmanned aerial vehicles 20 are operable to communicate with a single third-party system 42, intermediary server 44, home station 30, cloud 40, computing device 26, unmanned aerial vehicle 20, or a combination thereof. In another refinement, a plurality of unmanned aerial vehicles 20 are operable to communicate with one or more third-party systems 42, intermediary servers 44, home stations 30, clouds 40, computing devices 26, other unmanned aerial vehicles 20, or a combination thereof. It should be appreciated that the present invention is not limited by the number of targeted individuals 16, sensors 18, unmanned aenal vehicles 20, communication links 24, communication links 32, home stations 30, intermediary servers 44, third-party systems 42, clouds 40, computing devices 26, or other computing devices.

In another refinement, unmanned aerial vehicle 20 is operable to communicate with another one or more unmanned aerial vehicles 20. Communication amongst a plurality of UAVs may occur within a network or plurality of networks. Advantageously animal data and other information can be shared across UAVs within a single network or a plurality of networks. In addition, one or more home sensor duties can also be shared between UAVs (e.g., UAVs taking different actions related to the same animal data, same sensor, same home station, or same endpoint). In another refinement, an intra-UAV communication network can be created, with one or more unmanned aerial vehicles 20 acting within a single network or independently of each other, with one or more home stations communicating with one or more unmanned aerial vehicles 20, and two or more unmanned aerial vehicles 20 communicating with each other. In a variation, two or more unmanned aerial vehicles opeiate within a network, with one or more home stations operable to communicate with the network, and two or more unmanned aerial vehicles operable to communicate with each other. In another refinement, an inter-UAV communication network can be created, with two or more groups of unmanned aerial vehicles 20 acting within a single network or a plurality of networks. In another refinement, communication between the one or more UAVs and one or more third parties 42, intermediary servers 44, home servers 30, clouds 40, computed devices 26, unmanned aerial vehicles 20, or a combination thereof, can occur simultaneously.

In a variation, one UAV 20 may act as a primary server and data collection point with one or more other UAVs 20 acting as extensions of the primary UAV within a single network or plurality of networks. In another variation the one or more UAVs communicate solely with a primary UAV which in turn communicates with the one or more other computing devices (e.g., home station intermediary server, third-party) on behalf of all UAVs within a given network. Similar to a master/slave configuration, a UAV network may consist of a UAV “parent” that controls the other UAV “children” and at least a portion of their functions. In this example, the home station or system requesting data may only communicate with the primary UAV, with the primary UAV providing one or more instructions to the one or more other UAVs related to the one or more tasks or actions required. For example, in a cellular network, one or more UAVs may he utilized as an extension of an existing network whereby the one or more “children” UAVs follow a primary “parent” UAV to provide communications support related to the one or more sensors (e.g., functionally similar to a base station). In these scenarios, the UAVs may also be operable to provide communications support for non-animal data signals. In another example, the “children” UAVs may send all relevant sensor data collected from the one or more targeted individuals to the “parent” UAV, which then communicates to the relevant sensor data to one or more computing devices from a single UAV source.

In a refinement, a network consisting of at least one home station, at least one sensor, and at least one unmanned aerial vehicle is operable to monitor one or more characteristics related to the at least one sensor, the at least one unmanned aerial vehicle, electronic communication within the network collected animal data distribution of the collected animal data, or a combination thereof In another refinement, the network includes one or more intermediary servers, third-party computing devices, cloud servers, othei computing devices, or a combination thereof. In another refinement, two or more unmanned aerial vehicles operate within the network, with one or more home stations operable to electronically communicate with the two or more unmanned aerial vehicles as part of the network, and two or more unmanned aerial vehicles operable to electronically communicate with each other. In this case, electronic communication can include providing animal data from one unmanned acnal vehicle to another one or more unmanned aerial vehicles In another refinement, the two or more unmanned aerial vehicles execute one or more coordinated actions in response to one or more commands The one or more commands may be pre-programmed or provided by the home station or other computing device. In a refinement, the one or more commands may be generated utilizing one or more artificial intelligence techniques. Commands may include one or more actions taken upon the data by the two or more UAVs, location of the data being sent, formation changes amongst the UAVs, and the like.

In another refinement, one or more unmanned aerial vehicles 20 take one or more coordinated actions on at least a portion of the same data. For example, UAV1 may collect the animal data and attach metadata, and UAV2 may access the animal data from UAV1 and take one or more processing steps on the collected animal data with its associated metadata. In another variation, one UAV may collect the animal data and attach metadata to the collected animal data, and send at least a portion of the collected animal data to another UAV to take one or more actions (e.g., one or more processing steps) while storing at least a portion of the collected animal data, which can he utilized by the UAV or provided (e g made available for download, sent) to one or more computing devices at a later time. In another variation, one UAV may collect the animal data and take one or more actions on the collected animal data (c g., attach metadata), and send at least a portion of the collected animal data to another UAV to take another one or more actions on at least a portion of the collected animal data (e g. analyze the data, store the data) while providing (e.g., sending, making available) at least a portion of the collected animal data to one or more third parties (e g., by sending it directly to a third party, making the data available via cloud 40, and the like).

In a variation, at least one of unmanned aerial vehicle 20, home station 30, intermediary server 44, cloud server 40, or computing device (e.g., computing device 26) are operable to assign one or more classifications to the animal data, the one or more classifications including at least one of computed asset classifications, insight classifications, targeted individual classifications, sensor classifications, unmanned aerial vehicle classifications, data property classifications, data timeliness classifications, or data context classifications. Classifications (e.g., groups, tags) can be created to simplify the search process for a data acquirer, provide more exposure for any given data provider or data set by categorizing data for simplified access to relevant data, and the like. Classifications can be based may be based on targeted individual-related characteristics, sensor-related characteristics, data collection processes, practices, associations, and the like. Examples of classifications include computed asset classifications (e g, properties of the targeted subject captured by the one or more sensors that can be assigned a numerical value such as heart rate, hydration, etc.), targeted individual classifications (e.g. age, weight, height, medical history), insight classifications (e.g., “stress,” “energy level,” likelihood of one or more outcomes occurring), sensor classifications (e.g., sensor type, sensor brand, sampling rate, other sensor settings) UAV classifications (e.g., UAV type, settings, characteristics), data property classifications (e.g., raw data or processed data), data quality classifications (e.g., good data vs. bad data based upon defined criteria), data timeliness classifications (e.g., providing data within milliseconds vs. hours), data context classifications (e.g., NBA finals game vs. NBA pre-season game), data range classifications (e.g., bilirubin levels between 0.2 – 1.2 mg/dL), and the like. Additional details of classifications and their association with the animal data, as well as monetization systems for animal data, are disclosed in U.S Pat. No. 62/834,131 filed Apr. 15, 2019, U.S. Pat. No. 62/912,210 filed Oct. 8, 2019, and U.S. Pat. No. PCT/US20/28355 filed Apr. 15, 2020, the entire disclosures of which is hereby incorporated by reference.

In another variation, in the event UAV 20 is unable to electronically communicate with home station 30 or third-party system 42, intermediary server 44, computing device 26, or cloud 40, or has been instructed to not provide (e.g., transmit/send, make available) collected animal data to any computing device, unmanned aerial vehicle 20 may continue to collect and store animal data (e.g., locally on the UAV, in cloud 40 it available, or a combination thereof). In this scenario, the collected animal data can be transmitted when the connection to home station 30, third-party system 42, intermediary server 44 computing device 26, or cloud 40 is reestablished, or when the one or more UAVs have been instructed to do so. In another variation, the one or more UAVs mav be instructed to send at least a portion of the collected animal data from the one or more sensors to a third-party, intetmediary server, or home station while storing the collected animal data (e.g., locally on the UAV, in cloud 40, or a combination thereof) for possible use at a later time. In yet another variation, if the unmanned aerial vehicle is not in electronic communication with the at least one sensor, the unmanned aerial vehicle is operable to initiate electronic communication (e.g., reconnect) with the at least one sensor after one or more of the following parameter changes time, one or more chatacienstics of the at least one sensor (e.g., location, sensor parameters), one or more characteristics of the at least one targeted individual (e.g., location, elevation, its connection with one or more other computing devices such as its cloud server), or one or more characteristics of the one or more unmanned aerial vehicles (e.g., location, elevation). Reconnection may occur automatically location can include physical location or ditectional location of the UAV or any of its components (e.g., the direction a transmission component on the UAV is pointing like an antenna or beam pattern). It should be appreciated that such parameters are merely exemplary and not an exhaustive list. Depending on the scenario, one or more other parameters may be deemed more relevant than others. Additionally, automatic reconnection between the one or more UAVs and the one or more sensors or one or more other end points may occur via an instruction or series of instructions (e.g., a control command) sent from the home station, or instruction or series of instructions that are programmed (e.g., pre-programmed or dynamic based upon one or more artificial intelligence techniques) on the UAV (e.g., the UAV is progiammed to automatically reconnect). The collected animal data can be provided (e.g., transmitted) when the connection to the home station or third-party system or intermediary server is reestablished.

In a refinement, home station 30 can be operable to take on a number of different roles. For example, home station 30 can be operable to set up (e.g. configure) one or more sensors 18, provide one or more commands to one or more sensors 18 and/or UAVs 20 (e.g. commands to take one or more actions on the data such as distribute the data), transform sensor data collected by UAV 20 (e.g., analyze the data, visualize the data), monitor the one or more sensors 18, UAVs 20, and/or the one or more networks that include one or more sensors 18 and UAVs 20, and the like For example, home station 30 may be a telehealth or health monitoring application whereby one or more sensors 18 are piovided one or more commands by home station 30 and paired with the one or more UAVs 20, with animal data being sent back to home station 30 via one or more UAVs 20 for visual display. In a refinement, home station 30 is operable to provide one or more commands to the one or more UAVs for data distribution In another refinement, home station 30 monitors the one or more UAVs and/or at least one sensor, with an event occurring that either (1) alerts the one or more home stations 30, intermediary servers 44, third-parties 42, UAVs 20 (including one or more other UAVs 20), computing devices 26, or a combination thereof, and/or (2) prompts the home station to take at least one action (e.g., collective action) in furtherance of delivering an expected output to the one or more home stations 30, intermediary servers 44, third-parties 42, computing devices 26, UAVs 20, or a combination thereof For example, the system may be capable or monitoring the one or more UAVs and take one or more corrective actions ielated to error conditions and failure. If the connection between sensor and UAV is weak or the UAV has an energy-related issue (e.g., power issue such as battery degradation), a corrective action instructed by the home station can be for a replacement UAV to travel and replace the faulty UAV being utilized to transmit from sensor to home station 30, cloud 40, intermediary server 44, third-party 42, or other computing devices. In a variation, the home station may instruct the LAV being replaced to communicate with the replacement UAV to ensure it is taking over specific UAV-related duties for that UAV (e g, connecting with a specific sensor(s), being a relay hub in a specified network, etc.) and provide the new UAV with access to the relevant information (e.g., collected animal data historical data, algorithms, integrations with data endpoints) to ensure the replacement is seamless. Access to relevant information may occur either directly (e.g., communication between LAV and UAV; communication between UAV and home station) or via communication with cloud 40. In another example, if a UAV determines an issue with the at least one sensor (e.g., sensor connection is faulty, the sensor is bad) and the UAV cannot connect or reconnect, an automated action may occur whereby a backup UAV is deployed to connect with the one or more sensors, and/or the UAV sends an alert to the home station to take one or more actions related to the sensor (e.g., an alert to replace the one or more sensors). In yet another example, the UAV may detect a health or medical condition based upon the collected animal data, which may tugger either an alert or another action bv the UAV (e.g., sending at least a portion of the collected animal data) to the one or more home stations, intermediary devices, or third-parties. The one or more home stations may utilize one or more artificial intelligence techniques (e.g., machine learning, deep learning) to calculate, compute, derive, extract, extrapolate, simulate, create, modify, enhance, estimate, evaluate, inter, establish, determine deduce, observe, communicate the one or more actions In another refinement, the one or more UAVs 20 are programmed to dynamically take the at least one corrective action in furtherance of delivering an expected output to the one or more home stations, intermediary servers, or other computing devices (e.g., third-party systems, UAVs, and the like). In another refinement, one or more alerts are provided by the unmanned aerial vehicle to a computing device (e.g., home station, intermediary server, third-party) in response to the received animal data including its one or more derivatives. One or more actions are taken by the home station or unmanned aerial vehicle based upon the one or more alerts In another refinement, one or more alerts are provided by the unmanned aerial vehicle to a computing device (e g, home station, intermediary servet third-party) in response to information derived from the one or more actions taken by the one or more UAVs with at least a portion of the collected animal data.

In a refinement one or more artificial intelligence techniques may be utilized either directly or indirectly (e.g. via their associated cloud) bv home station 30, computing device 26, and/or UAV 20 to dynamically provide one or more commands to send the data to one or mote computing devices based upon the collected animal data (e.g., inclusive of its one or more derivatives). For example, it the collected animal data demonstrate one or more irregular readings, the data may be sent to a medical professional or medical system (e.g., hospital) for further examination. In another refinement, the animal data is provided to one or more endpoints (e.g., third-parties, computing devices utilized by targeted individuals) for consideration. In this regard, one or more stakeholders 47 may receive considerationfor the animal data (e.g., payment, and/or a trade for something of value). For example, in the event an insurance company receives the animal data via the one or more UAVs either directly or indirectly (e.g., via the cloud in communication with the one or more UAVs, or a via a third-party that receives the animal data via the cloud in communication with the one or more UAVs), consideration may be provided to a stakeholder (e.g., an insurance premium for the one or more targeted individual stakeholders may be adjusted). A stakeholder can be any individual, group of individuals, a corporation, and the like who or which has a commercial right to the collected animal data, including its one or more derivatives. For example, the targeted individual can be a stakeholder for their own animal data and a basketball team can be the stakeholder for the animal data for their targeted group (i.e., entire team) of targeted individuals (i.e., the players), a third-party company that has obtained the rights to the animal data from a targeted individual or group of targeted individuals, and the like In a refinement, consideration may be non-monetary in nature so long as it has value to one or both parties. For example, a targeted individual stakeholder may agree to provide a third-party entity (e.g., analytics company) with their animal data in exchange for obtaining animal data insights related to their own body (e.g., real-time health vitals, predictive health insights) This transaction can be monitoted by home station 30 intermediary server 44, or other computing devices (e.g., 3^{rd} party monitoring systems). In another example, a targeted individual may consent to provide (and enable use of) their sensor data to a healthcare company via the one or more UAVs directly or indirectly (e g, via the cloud in communication with the one or more UAVs), in order for the healthcare company to monitor the vitals of the targeted individual and take one or more actions (e.g., notify a medic, send an ambulance to their location) should there be an abnormality in the one or more readings.

It should be appreciated that the one or more unmanned aerial vehicles 20 can be operable to electronically communicate with each other, exchange information (e.g., sensor signal information, metadata), and execute one or more coordinated actions as part of a network or plurality of networks. In a refinement, one or more unmanned aerial vehicles 20 can serve to support sensor communication (e.g., extend existing sensor communication) and/or animal data access opportunities between one or more sensors 18 and one or more home stations 30, computing devices 26, intermediary servers 44, third-parties 42, clouds 40, or a combination thereof. For example, one or more UAVs can be utilized within a network to support communication-related functions from home station 30 to one or more sensors 18 located on one or more targeted individuals 16, or from the one or more sensors 18 to one or more computing devices (e.g., third-party computing device 42, intermediary server 44). In the event one or more sensors are prevented from communicating with the one or more computing devices (e.g., distance between the sensor and system, line of sight issues, sensors have limited communication range or capabilities), one or more UAVs may be used to extend the communications between sensor and computing device and/or provide more reliable electronic communication links. In another refinement, one or more UAVs can he used to support existing networks (e.g., UWB-based communication networks, cellular systems). For example, by utilizing a UAV’s mobility, as well at its ability to establish a more direct line-of-sight with any given targeted individual, an existing network (e.g., on-ground network) can create wider coverage and enhance the overall performance of the network (e.g., including delay latency issues, coverage, and the like). In another example one or more UAVs or network of UAVs may be utilized to provide coverage in “dark areas” for cellular networks.

In a refinement, the one or more electronic communications between at least one sensor 18 and home station 30 is transferred (e.g., “handed off”) from a non-unmanned aerial vehicle computing device to one or more other UAVs, and vice versa, within a network or a plurality of networks For example, if a sensor is commumcating with home station 30 via a computing device’s internal communications system (e.g., smartphone using Bluetooth) and the computing device ceases transmission from a sensor to the home network, the one or more UAVs may be operable to detect that a connection has been lost between sensor and computing device (e.g., via an alert) and initiate communication between sensor and UAV, enabling the UAV to serve as an extension between the sensor and the home station, intermediary server, third-party, or other computing device Once the sensor has been “handed off” from the computing device to the one or more LAVs, the home station may update the one or more UAVs with all required characteristics related to the sensor, the animal data, the targeted individual, the required one or more actions, the required one or more outputs, and/or the required one or more distribution points. An update may occur via direct communications links or indirectly (e.g., providing access to the data via cloud 40) In a variation, the UAV can be programmed to “hand off” sensor communication to one or more computing devices in the event the sensor is not able to communicate with the UAV (e.g., if the individual is in an area where there is no signal communication with the UAV). In another refinement, one or more artificial intelligence techniques may be utilized to predict future signal communication issues (e.g., future communication cutoffs) based on data related to the one or more targeted individuals including their movement patterns (e.g., actual and/or predicted) historical data (e.g., historical UAV signal communication data based on actual and/or predicted movements), and the like.

In another refinement, the one or more “handoffs” between the home station and UAV, UAV-to-UAV, or UAV to other computing devices (e.g., home station) includes the transfer of at least a portion ot the collected information related to the targeted individual the sensor, and/or the collected animal data. To effectively serve each targeted user during the hand-off period (e.g., in order to maintain connectivity between the sensor and the UAV system), information related to the one or more targeted individuals and their corresponding sensor data may need to be shared and made available across multiple UAVs either via the home station, the cloud or shared from UAV-to-UAV Efficiencies can be extrapolated utilizing one or more artificial intelligence techniques to predict what data may need to be shared (or made available) or vs. stored, what information may need to be duplicated across computing devices to ensure a seamless handoff movements of the one or more targeted individuals (e.g., to ensure the hand-off process is with the correct UAV or network of UAVs), required or requested data outputs, and the like.

In another refinement, the one or more UAVs take one or more actions to enhance electronic communication between the one or more UAVs and one or more other sensors or computing devices (e.g., home station 30 another one or more UAVs 20). For example the mobility and aerial nature of the one or more UAVs enable the one or more UAVs to establish line-of-sight with one or more targeted individuals 16 to maintain electronic communication with one or more sensors 18, as well as other systems (e.g., home station 30, computing device 26, third-party system 42, intermediary server 44). In this regard, the one or more UAVs 20 may use one or more techniques (e.g., beamforming) to locus the one or more communications (e.g., wireless signals) towards the one or more sensors In a refinement, optimizing beam patterns and directions, as well as placement/formation of the one or more UAVs (e.g., including changes in altitude/elevation), may occur by utilizing one or more artificial intelligence techniques, which can use one or more actions (e.g., change in UAV formations, increase/decrease number of UAVs antenna positioning, type of antenna used, antenna array positioning) to achieve the desired result (e.g., maximize total coverage area ensuring reliable communications between sensor and UAV). In a refinement, optimizing beam patterns and directions, as well as placement/formation of the one as more UAVs, may occur dynamically utilizing one or more artificial intelligence techniques.

An example of unmanned aerial vehicle 20 in FIGS. 1 and 2 is an aircraft that is piloted by remote control or autonomous onboard computers without a physical onboard human presence (i.e., a person’s body). Examples of such aircraft include a high-altitude long-endurance aircraft, a high-altitude pseudo satellite (HAPS) an atmospheric satellite, a high-altitude balloon, a multirotor drone, an airship, a fixed-wing aircraft, or other low altitude systems. More specific examples of fixed-wing aircraft include high-altitude long-endurance (HALE) aircraft, multirotor aircraft, and other fixed-wing aircraft. Other names used interchangeably with UAV include RPAS (Remotely Piloted Aircraft System) and Drone. In the case of UAVs such as HALE or HAPS aircraft, which typically fly at high altitudes (e.g., in Class A airspace over the U.S. - 18000 ft MSL to 60,000 ft MSI - or near orbital) above most weather and commercial air traffic, they are oftentimes designed for long flight times, ranging from 3-4 months at a time (or longer) without landing. Some UAVs have the capabilities to stay aloft for years or longer without the need to land ot refuel (e.g., solar powered aircraft). Examples include geostationary balloon satellites or other plane-based atmospheric satellites. Another example of a UAV is a multirotor aircraft. Multirotor aircraft are popular types of drones and have two or more rotors. The rotors allow it to function as a helicopter. In yet another example ot a UAV, a VTOL (vertical take-off and landing) aircraft can take off and land vertically with the ability to hover mid-flight. VTOL unmanned aerial vehicles, are most commonly of the multirotor design. Some newer VTOLs are hybrid multirotor/fixed-wing aircraft that can take off and land vertically using multiple rotors but then transition to horizontal flight using wings and a propeller.

Additional specific examples of drones include, but are not limited to, Airbus Zephyr S, Airbus Zephyr T, Aurora Odysseus, Raven Aerostar STRATOSPHERIC AIRSHIPS, Raven Aerostar THUNDERHEAD BALLOON SYSTEMS, AeroVironment HAWK30, AeroVironment Global Observer, Astigan A3, General Atomics MQ-9 Reaper, Ukrspecsystems PC-1 Multirotor Drone, Ukrspecsystems PD-1 VTOL, Ukrspecsystems PD-1 Fixed Wing, DJI Mavic 2 Enterprise Drone, and DJI Phantom 4 Pro V2.0.

In addition, the elevation at which the unmanned aerial vehicle can “sit” (e.g., hover) and/or glide can vary. For example, the unmanned aerial vehicle can be a high-altitude pseudo satellite, which flies in high altitudes above weather and commercial air traffic. These aircraft are typically designed for long flight times (e.g., which can range from 3-4 months at a time to much longer without landing). UAVs such as a HAPS can also be utilized as one or more communication links between other UAVs (e.g., drones) flying close to the earth’s surface or another system and satellites orbiting in space. This may be advantageous in the event one type of UAV has different capabilities (e.g., computing capabilities) than other UAVs. Additionally, one or more UAVs such as HAPS can be useful as an intermediate relay step between a satellite and a ground station, supporting the transfer of sensor data and reducing the ground and satellite infrastructure required. HAPS can efficiently complement the one or more networks where the target area is limited and changing, as well as where ground infrastructure is nonexistent or unavailable.

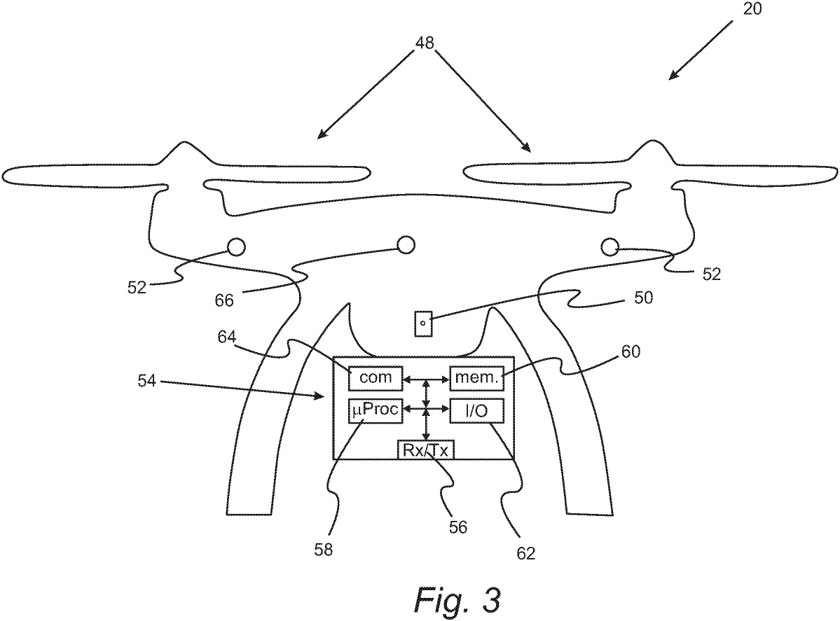

FIG. 3 depicts a schematic of a variation of an unmanned aerial vehicle that can be used in UAV-based transmission system 10 and 10′. Note that the present invention is not limited by the types of UAVs that can be utilized in UAV-based transmission system 10 and 10′. Unmanned aerial vehicle 20 includes an aerial propulsion system 48 that may vary based upon the particular design or method of flight of the unmanned aerial vehicle. In a refinement, unmanned aerial vehicle 20 includes one or more optical sensors 50 (e.g., cameras) which can be used to capture visual information (e.g., one or more videos, photographs, streamed media) of one or more targeted individuals 16, other one or more individuals (e.g., spectators), and/or the proximate area thereof. In a variation, the one or more optical sensors 50 may be accompanied by audio and/or other data from the UAV and its one or more sensors. Advantageously, the information collected by the one or more UAVs may be provided (e.g., sent, accessed, streamed) in a real-time or near-time capacity (e.g., in the event the one or more UAV camera sensors are utilized to send a streaming feed for a live sports broadcast; in the event a military organization wants to the track the location of its soldier via real-time video; in the event a UAV operator requires a real-time or near real-time video feed to pilot/navigate the one or more UAVs) or may be provided to one or more endpoints at a later time. Optical sensor 50 may also be controlled via the control application on Home Station 30 or on another computing device. In a refinement, unmanned aerial vehicle 20 can include one or more other sensors 52 which may include another one or more optical-based sensors (e.g., in the event multiple optical sensors are required for different visual-based functions), weather sensors (e.g., wind speed, temperature, humidity, barometric pressure), or other sensing technologies relevant (either directly or indirectly) to one or more targeted individuals 16 (e.g., sensors to track targeted individuals to track and record physical distances between targeted individuals to ensure they are effectively social distancing, with one or more alerts being provided to notify any failures in distancing). Advantageously, the one or more sensors 50 and 52 may be modular in nature, enabling the UAV to “swap out” sensors at any given time. Unmanned aerial vehicle 20 includes a data acquisition unit 54 that electronically communicates with the at least one sensors 18. In some variations, the data acquisition unit 54 includes a microprocessor 58, which is in electronic communication with memory module 60 and input/output module 62. Microprocessor 58 can be operable to execute one or more data processing steps. In a refinement, the data acquisition unit includes transceiver module 56 that electronically communicates with one or more sensors 18. The transceiver module 56 includes one or more antennas that may be part of an antenna array on a single UAV or across multiple UAVs. This communication is typically via a two-way communication link in which a user can activate and set parameters for the one or more sensors and received data signals from the one or more sensors. In a variation, the data acquisition unit includes a transceiver module 56 operable to send one or more commands to the at least one sensors and receive one or more data signals or readings from the at least one sensor. In another variation, communication may be one-way (e.g., one or more of the UAVs may only be configured to receive information from the sensor and not configured to send commands or data to the sensor). In a refinement, the one or more transceiver modules 56 enable the one or more UAVs to communicate with one or more sensors 18 and another one or more computing devices (e.g., home station 30, other UAVs 20, computing device 26, cloud 40) as part of a wireless mesh network. In another refinement, data acquisition unit 54 includes a communication module 64 which allows communication via one or more protocols (e.g., Ant+, BLE, LoRa, ultra-wideband, WIFI, cellular, and the like). In some variations, communication module 64 can be operable to enable a plurality of simultaneous connections and communications protocols. In another refinement, transceiver module 56 can be configured to communicate with one or more home stations 30, third-party systems 42, intermediary servers 44, computing devices 26, clouds 40, or a combination thereof. In another refinement, transceiver module 56 is operable to receive one or more signals from the source of animal data and to send one or more control signals to the source of animal data (e.g., inject more insulin, turn off a sensor, stream data when a command is given, and the like). Typically, the ability to send one or more control signals will be a command via a control application from the one or more home stations 30. In a refinement, one or more artificial intelligence or statistical modeling techniques are utilized to create, enhance, or modify one or more actions taken by the one or more unmanned aerial vehicles 20. For example, one or more artificial intelligence techniques may be utilized to autonomously create and send one or more control signals based upon the collected animal data. This may occur on home station 30, the one or more UAVs 20, or another computing device. In another refinement, transceiver module 56 is operable to communicate with (e.g., send one or more signals, receive one or more signals) one or more non-animal data sources. In another refinement, transceiver module 56 is operable to provide (e.g., send) and receive at least a portion of non-animal data related to the one or more targeted individuals (e.g., non-animal cellular data from the one or more targeted individuals).

In a refinement, the one or more unmanned aerial vehicles take one or more actions in response to one or more calculations, computations, predictions, probabilities, possibilities, estimations, evaluations, inferences, determinations, deductions, observations, or forecasts that are derived from, at least in part, the received animal data. One or more actions may be taken based upon one or more instructions provided by home station 30 or from instructions provided by one or more UAVs. In some cases, one or more calculations, computations, predictions, probabilities, possibilities, estimations, evaluations, inferences, determinations, deductions, observations, or forecasts can include non-animal data. In a variation, the one or more actions include at least one of providing one or more alerts to one or more computing devices or providing animal data to one or more computing devices that generate one or more alerts based upon the animal data. In some cases, one or more artificial intelligence or statistical modeling techniques are utilized to create, enhance, or modify the one or more actions taken by the one or more unmanned aerial vehicles. In a refinement, one or more actions are taken by the one or more unmanned aerial vehicles or computing devices in response to the one or more alerts In some cases, one or more artificial intelligence or statistical modeling techniques are utilized to create, enhance, or modify one or more actions taken by the one or more unmanned aerial vehicles or computing devices.

In another variation, one or more unmanned aerial vehicles 20 have attached to, affixed to, integrated with, or embedded within, at least one sensor 66 that captures one or more signals or readings (e.g., wind speed temp, humidity UV%, animal recognition, video data location via optical tracking elevation, other audiovisual information). Sensor 66 can also be a tracking system for the one or more targeted individuals (e.g., RFID-based location tags located on sensor 18 or computing device 26 if local to the targeted individual and operable to be tracked by the one or more UAVs). In a refinement, at least a portion of the one or more signals or readings are provided by the one or more unmanned aerial vehicles to another computing device. In another refinement, at least a portion of the one or more signals or readings are used by the one or more unmanned aerial vehicles to transform collected animal data into one or more computed assets or insights. In a variation, utilizing the one or more signals or readings captured from the at least one sensor 66, which may include animal data, non-animal data, or a combination thereof, the one or more UAVs 20 are programmed to at least one of (1) take one or more actions utilizing the captured sensor data (e g., utilize data from sensors 18 and 66 to transform the captured data into a computed asset or insight), and (2) provide at least a portion of the sensor data to the home station 30, intermediary server 44, third party 42 computing device 26 cloud 40 or a combination thereof For example, the UAV may have attached, affixed, or embedded sensors that utilize facial recognition software to capture emotion from one or more targets. The UAV may then utilize the collected data from one or more sensors 18 on the targeted individual to correlate the collected facial recognition data with the sensor 18 animal data (e.g., ECG data that can provide heart rate variability) to determine the “stress” level of an individual at any given time. In another example, one or more UAVs may be utilized to enable more effective social distancing by tracking the location of targeted individuals taking one or more precessing steps related to the data to determine the distance between targeted individuals at any given time, and relay an alert to home station 30, computing 26, or other computing devices local to the targeted individuals notifying them of any physical-distancing related issues that arise (e.g., one targeted individual has moved into a space that is within n number of feet of another targeted individual). In a refinement, the signals or readings from sensor 66 can include both animal and non animal data. In another refinement, sensor 66 is sensor 52. In another refinement, sensor 52 is sensor 66.

In a refinement, the one or more unmanned aerial vehicles take one or more actions (e.g., collect data) based upon, at least in part, the animal data (e.g., signals or readings of the one or more sensors). In some cases, one or more actions taken may utilize information gathered from non-animal data sources. For example, if a targeted subject’s respiration rate reaches a predetermined level, the one or more sensors on the one or more UAVs may be programmed to start collecting data via the one or more optical trackers (e.g., record video from the camera) for a period of time and/or until the targeted subject’s one or more biological readings return back to a predetermined level or threshold. The information may be sent directly to a third party or sent to the cloud from which the information can be accessed. In a variation, the one or more predetermined levels may be changed or modified based on one or more factors, which may include the activity of the individual (e.g., is the subject engaging in an activity that is causing the biological readings to increase) and the like. In another refinement, the one or more sensors on the one or more UAVs may collect data, which may be intermittent or periodic, and take one or more actions based upon the one or more biological readings. For example, if a targeted subject’s respiration rate reaches a predetermined level, the one or more UAVs may collect visual information via an optical sensor (e.g., optical camera/tracker), take one or more actions by analyzing the collected visual information to determine the cause of the increased or decreased respiration rate, and taking a further one or more actions based upon the determination (e.g., the analysis of the captured video may conclude that the subject has changed activity so the UAV takes no action; the analysis of the captured video may conclude that the subject is having a medical episode so the UAV contacts medical help; the analysis of the captured video may conclude that the UAV should start recording the targeted individual via the optical sensor). In a variation, one or more artificial intelligence techniques may be utilized to make a conclusion or determination related to the one or more biological readings and the information captured from the one or more sensors on the one or more UAVs (e.g., optical trackers such as an optical camera).

In another refinement, the one or more UAVs mav act as a remote health system (e.g., first aid kit) and utilize at least a portion of the animal data from one or more targeted individuals 16 to identify one or more issues (e.g., the sensor readings indicate the person is having heart failure or heart stoppage) and take one or more actions. The one or more UAVs may contain or transport equipment, (e.g., defibrillator), medication, or other aid (e.g., sensing equipment to more closely monitor the targeted individual) that enables the one or more UAVs to travel to the location to the one or more targeted individuals so that aid can be administered (e.g., a person with the targeted individual may access the defibrillator located on or brought by the UAV in order to administer it on the targeted individual who is experiencing heart failure). In the event, the UAV contains sensors and/or sensing equipment integral to, or part of, the UAV or carried by the UAV, the UAV may be operable to relay information captured by the UAV to the one or more other UAVs within the network or to other computing devices (e.g., third party 42, which may be a hospital or EMT). In a variation, multiple UAVs may be utilized, with each UAV carrying out a task or multiple tasks related to the action (e.g., one UAV may collect and analyze the sensor data, and provide the alert related to the heart failure; another UAV may be sent out to the targeted individual with the medical equipment to enable administration of aid).

It should be appreciated that the one or more actions taken by UAV-based transmission system 10 and 10′ can include processing (e g. transforming) acquired animal data into a form for distribution and in particular, perhaps (but not necessarily) into a form for consumption (e.g., monetization). In this regard, data acquisition unit 54 collects the animal data from one or more sensors 18. Data acquisition unit 54 is operable to execute one or more data processing steps such as, but not limited to normalizing, time stamping, aggregating, storing, manipulating, denoising, enhancing, organizing, analyzing, anonymizing, summarizing, synthesizing, bookkeeping, synchronizing and/or distributing the animal data. By utilizing a UAV-based system, distribution can occur over longer distances from the at least one sensor to another computing device (e g., home station, a third-party system such as a user watch, user phone, health, and wellness system, media/sports betting system).

With reference to FIGS. 1 and 2, UAV-based transmission system 10 or 10′ can communicate in real-time or near real-time with one or more home stations 30, third-parties 42, intermediary servers 44, computing devices 26 clouds 40, or a combination thereof. Such real time or near real-time communication may occur, for example, during an activity such as a sporting event. In this example, one or more third-parties 42 can be a media platform (e.g., broadcaster) of a sporting event or an application utilized by spectator (e.g., a sports betting application). Other third-parties 42 that would benefit from real-time or near-time sensor communication include a system utilized by coach/ trainer for monitoring physical activity, an airline system to monitor pilots, a hedge fund system to monitor traders, an industrial system (e.g., construction site, assembly line) to monitor workers, a hospital monitoring system for outpatients, a military monitoring system for soldiers, an insurance company system for monitoring the individuals it insures, a telehealth or wellness platform for monitoring its users, and a variety of other use cases. In a refinement, UAV-based transmission system 10 or 10′ can operate as an intermediary computing device (e.g., intermediary hub) in which the UAV 20 collects and distributes sensor data. In a variation the computing device (e.g., home station) executes a control application that provides one or more commands to the one or more unmanned aerial vehicles, the one or more commands initiating at least one of: (1) activating one or more sensors; (2) steaming at least a portion of collected animal data one or more computing devices (e.g., back to the control application, intermediary server, or third-party; (3) selecting one or more data streams to be sent to one or more computing devices (e.g., control application, ititeimedeary server or third party); (4) selecting a hequency upon which animal data is sent to one or more computing devices (e.g., back to the control application, intermediary server, or third party); (5) taking one or more actions upon collected animal data, and sending actioned upon data to one or more computing devices (e.g., the contiol application intermediary server, or third party); (6) changing or adjusting one or more settings within the at least one sensor; (7) taking one or more actions bv the at least one sensor; (8) changing or adjusting one or more characteristics (e.g., location. UAV sensor direction, antenna direction) of one or more unmanned aerial vehicles (e.g., including its one or more components); (9) taking one or more actions based upon information derived from the animal data (e.g., providing medical support via the UAV); (10) supporting (e.g., extending) electronic communication between one or more home stations and one or more other computing devices (e.g., home station, third-party systems, intermediary servers, other computing devices); or (11) storing sensor data. One or more actions upon the collected animal data can include one or more processing steps (e.g., transforming at least a portion of the collected animal data on the unmanned aerial vehicle into at least one computed asset or insight, with at least a portion of the collected animal data originating from the at least one sensor, and sending the at least one computed asset or insight to another computing device such as the control application, intermediary server, or third party, aggregating two or more data streams received by the unmanned aerial vehicle to create one or more insights that can be sent back to another computing device such as the control application intermediary server, or third party). It can also include any one or more actions taken with the data (e.g., the act of sending the data to another computing device).

In some variations, the control application is operable to set up and/or control at least a portion of UAV functionality, as well as manage (e.g., administer) the network that includes at least one sensor, at least one home station, and at least one UAV Depending on the unmanned aerial vehicle or the established network of UAVs, the LAV or network of UAVs may be operated via single control application or multiple control applications; one or more of which may be remote. In a refinement, once communication is established between the at least one sensor and UAV or network of UAVs, the UAV or network ot UAVs can act in one or more of four roles: (1) as an extension of the home station to facilitate communication between the one or more sensors and the home station or one or more other computing devices (e.g., third-party computing devices); (2) as an intermediary computing device that communicates with the one or more sensors, receives sensor data, and takes one or more actions (e.g., processes data, stores data sends data over longer distances to the home station or third-party computing device send commands provide support in response to information derived from the sensor data, such as on-ground support in the event of a medical emergency); (3) as one or more sensors that collects data related to (either directly or indirectly) one or more targeted individuals; (4) as an administrator of the one or more networks (e.g., acting as the home station; or (5) a combination thereof. One or more actions taken by the one or more LAVs with the collected animal data can include a capability to normalize, time stamp, aggregate, store, piocess, manipulate, enhance organize, analyze, anonymize, summarize synthesize, bookkeep, synchronize, distribute, or a combination thereof, as well as create and disseminate commands. For example the one or more UAVs may be operable to summarize data that is sampled at high frequency rates (e.g., collect data at 250-1000 hz per second or more and summarize the data to be sent 1x per second) and send the summarized data to accommodate any number of use cases or constraints (e.g., limited bandwidth or throughout constraints). Commands can be sent and received from the control application to the sensor via the UAV This means the control application can be operable to set up control, configure, and operate all connected sensors via communication through the UAV. Advantageously, commands can be sent and received from the control application to the UAV for any number of sensors that are in communication with the UAVs or network of UAVs. In a refinement, one or more commands can be created dynamically by the home station and/or one or more UAVs utilizing one or more artificial intelligence techniques For example if the one or more UAVs identify an irregularity in the animal data derived from the one or more sensors of a targeted individual, the home station and/or the one or more UAVs may dynamically to create a command to send the data to a third party (e.g., hospital healthcare provider, emergency system), or take another action. In another refinement, one or more UAVs can function as the intermediary server.

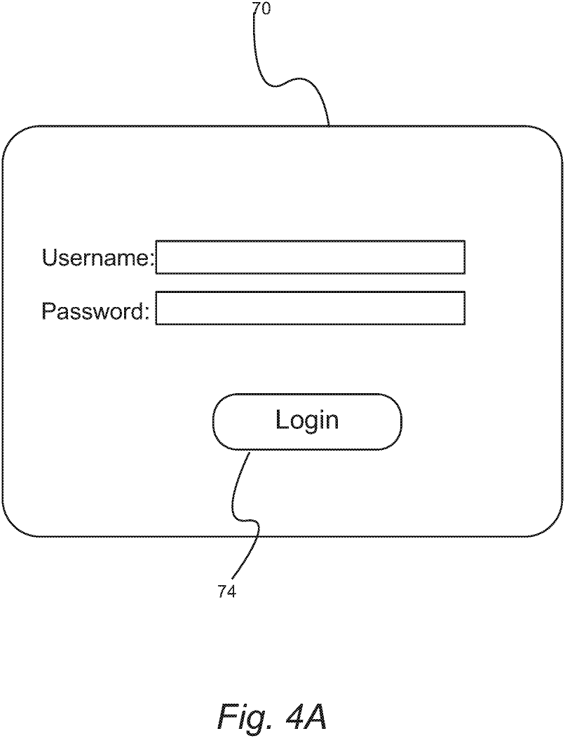

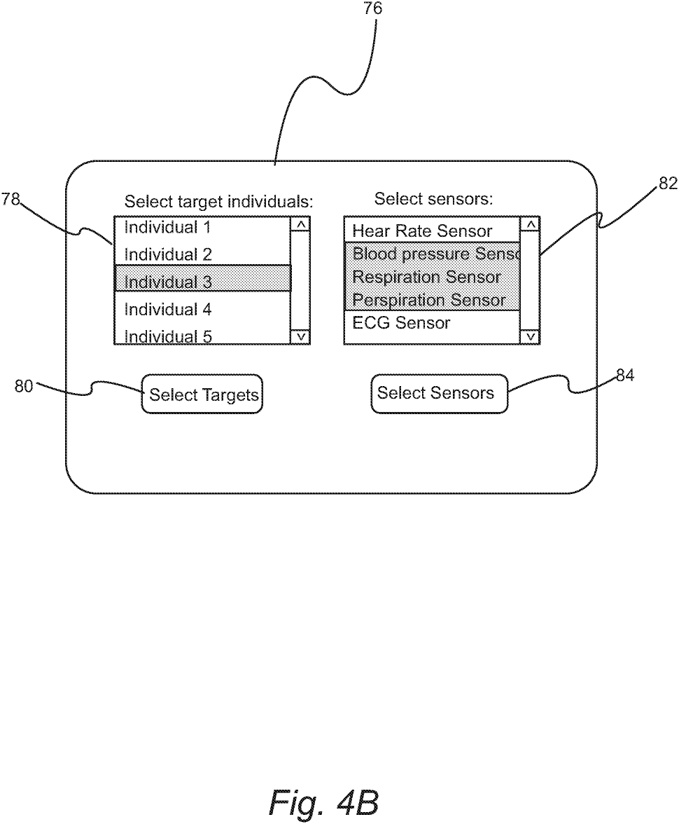

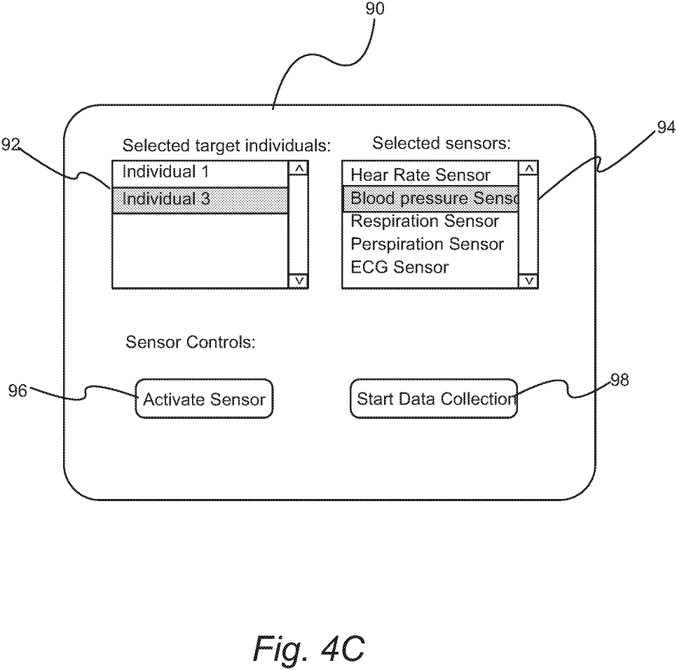

With reference to FIGS. 4A, 4B, and 4C, illustrations of a user interface for operation of the UAV-based transmission system 10 and 10′ are provided. It should be appreciated that for each of the control elements depicted in FIGS. 4A, 4B, and 4C, control elements such as selection boxes, dropdown lists, buttons, and the like can he used interchangeably. Each of the control elements in FIGS. 4A, 4B and 4C are depicted as “buttons.” A user logs into the control application via user interface 70 typically by entering a “username” and “password” and then actuating control element 74. Interface 76 is then presented to the user from which displays list box 78, show ing a list of targeted individuals that can he selected for monitoring. The user can select one or more individuals to monitor. In a refinement, one or more groups of targeted individuals (e.g., an entire basketball team all registered participants in an activity) may be operable for selection. Control element 80 finalizes the selection. The user then chooses at least one sensor from sensor list box 82 associated with the selected targeted individual (or group of targeted individuals). It should be appreciated that a sensor may capture more than one type of animal data. For example, a sensor that captures ECG may also have an accelerometer, gyroscope, and magnetometer in it to capture X,Y, Z coordinates. Control element 84 finalizes the selection.

With reference to FIG. 4C, after selecting the one or more targeted individuals and sensors user interface 90 is displayed. The user identifies which of the one or more selected sensors are to be operated. This is accomplished bv highlighting the selected one or more targeted individuals in list box 92 and the one or more sensors in list box 94. One or more control elements 96 power the sensor(s) “on” or initiate one or more commands (e.g., “get sensor ready”, “make available for wireless connection”, “pair”) if required. In a variation, control element 96 can be multiple control elements 96 if required. In a refinement, targeted individuals in list box 92 may be grouped together in one or more groups so that a user can select a category of users rather than individual users. Depending on the sensor the user can place the one or more sensor(s) on its form (e.g., body) if the user is a targeted individual, have the one or more sensors placed on the form if the user is a targeted individual and the like. Depending on the sensor the requirement related to when the one or more sensor are placed on the form (e.g., body) can be a tunable parameter. Some sensors may not icquire this step. The user then actuates a start data collection control element 98 for each sensor utilized by user to start collecting data.

Still referring to FIG. 4C, data collection between the one or more sensors and the control application can occur either before or after the selection of the one or more UAVs. In some cases, the one or more UAVs may be required for the data collection process to occur (e.g., whereby home station 30 can only communicate with sensor 18 via UAV 20). In other cases, the pairing of the one or more sensors 18 to the one or more UAVs 20 or networks of UAVs 20 can occur after the sensor has already started streaming to the control application on home station 30. In this case, a “hand off” in communication between home station 30 and UAV 20 may be required. In yet other cases, data collection may not start until the control application has successfully paired with one or more sensors and the one or more UAVs or networks of UAVs to ensure communication between the one or more sensors and the one or more UAVs or networks or UAVs is established with the control application receiving data only via the one or more UAVs. However, there are numerous ways in which data collection between the one or more sensors, home stations, and UAVs can occur, which have been previously described in detail. In a refinement, control elements 98 can be operable to start data collection for a subset, or all, or of the activated sensors. In another refinement, control element 96 can replace control element 98, and vice versa. For example, the control application may be configured to communicate with the sensor to automatically initiate data streaming from sensor to control application once the pairing function occurs between the sensor and the system (thereby eliminating any functional requirement for a “start data collection” control element).

In a variation, once the sensor is activated (or in some cases after the start of the data collection), the user can locate all UAVs (or a subset of relevant UAVs) within range of the one or more sensors and operable to connect with the one or more sensors, via the control application. Alternatively, the user can locate all UAVs within the parameters of the user activity (e.g., user is going on a 100 mile bike race which goes along a specific path), and select what UAV it would like to pair with. In another variation, the home station is operable to ping all UAVs within a network to automatically select and connect with the most optimal UAV (or network of UAVs) for the at least one sensor to connect with. This can be based upon the range of the one or more sensors and/or UAVs, pre-determined location pattern based on activity, and the like “Most optimal” can be defined in a number of ways depending on the use case including signal strength, mobility, bandwidth based on activity, and the like. In another variation, the one or more UAVs within a given location or range can automatically detect the at least one sensor and determine (e.g., via one or more artificial intelligence techniques) which UAV or network of UAVs the at least one sensor should pair with. In another variation, the home station can be operable to enable a user to select one or more networks of UAVs the one or more sensors can connect to. In another variation, the home station can be operable to automatically select the one or more networks of UAVs the one or more sensors can connect to. In yet another variation, the UAVs can be configured to enable the at least one sensor to switch or redirect communication from one UAV to another UAV if one or more parameter change related to the one or more sensors, targeted individuals, or UAVs (e.g., the one or more sensors or targeted individuals change location or another one or more issues that affect sensor or UAV communication arise such as signal strength or signal quality). In yet another variation one or more artificial intelligence can be utilized to determine the appropriate UAV or network of UAVs the one or more sensors should pair with.

In a refinement a home station is programmed to automatically select one or more unmanned aerial vehicles, or network that includes one or more unmanned aerial vehicles to connect with the at least one sensor based on one or more of following characteristics: unmanned aerial vehicle location, unmanned aerial vehicle coverage, unmanned aerial vehicle payload, unmanned aerial vehicle bandwidth network location, network coverage network payload, network bandwidth, targeted individual location, sensor location, energy constraints (e.g., battery life), signal strength, environmental conditions, or signal quality (e.g., including data packet loss). In a variation the home station or the one or more unmanned aerial vehicles provide one or more commands to the at least one sensor to take one or more actions (e.g., connect with another computing device such as another UAV, reduce sampling tate) based upon information derived from, at least in part, the one or more characteristics (i.e., the unmanned aerial vehicle location, unmanned aerial vehicle coverage, unmanned aerial vehicle payload, unmanned aerial vehicle bandwidth, network coverage, network payload network bandwidth, targeted individual location, sensor location, an energy constraint, signal strength, an environmental condition, signal quality, or a combination thereof). The one or more characteristics are detected and monitored by the home station or the one or more unmanned aerial vehicles in order to derive such information. In another refinement the home station can be programmed to automatically select the one or more networks of UAVs to connect with the at least one sensor based on one or more of following characteristics: network location, targeted individual location, data usage, network bandwidth, sensor location, energy constraints (e.g., battery life), signal strength, environmental conditions, or signal quality. In another refinement, a network consisting of at least one home station, at least one sensor, and at least one unmanned aerial vehicle is operable to monitor one or more characteristics related to the at least one sensor, at least one unmanned aerial vehicle, electronic communication within the network (e.g., sensor signal characteristics such as signal strength or signal quality), collected animal data, distribution of the collected animal data, or a combination thereof, and take one or more actions to maintain a determined level or quality of communication between the home station, the at least one sensor, the one or more UAVs one or more intermediary servers, one or more third-parties, one or more clouds and/or one or more other computing devices (e.g., computing device 26). For example, the network may provide an ability to monitor signal connection strength and signal quality across all potential data transfer points, as well as enable the home station to automatically select and change the best communication/data transfer point within the network, which may be another unmanned aerial vehicle, intermediary server, or other computing device. In a variation, the network may switch or redirect sensor communication from one UAV to another UAV based upon a desire to maintain or increase sensor signal strength and/or signal quality, as well as other relevant considerations including bandwidth availability, environmental conditions, energy constraints (related to the one or more UAVs), and the like. In another refinement, the at least one home station or unmanned aerial vehicle (1) detects and monitors one or more characteristics (e.g., signal quality, signal strength, UAV bandwidth) related to the one or more communications (e.g., sensor signals or readings) sent between the at least one sensor and one or more unmanned aerial vehicles across one or more data communication points (e.g., data transfer points), and (2) provides one or more commands to the at least one sensor to pair with (e.g, connect with) another computing device (e.g., secondary transmission source) which may be another UAV or other non-UAV transmission system. In a variation, the home station provides one or more commands that enables the at least one sensor to switch or redirect sensor communication from a UAV to another UAV based upon feedback provided to the home station or intermediary server by the one or more UAVs related to signal strength signal quality, UAV energy conservation (e.g., battery life), or other relevant considerations. Such switches or redirects may include alternating from one UAV to another.

In some variations, a network that includes a home station, at least one sensor and one or more unmanned aerial vehicles provides an ability to encrypt or compress data being stored or tiansnntted to or from the at least one sensor, home stations, or unmanned aerial vehicles In other variations, a network that includes a home station, at least one sensor, and one or more unmanned aerial vehicles is operable to encode (e.g., encrypt, compress, obfuscate) at least a portion of the animal data being provided (e.g., sent) to or by the one or more sensors, home stations or unmanned aerial vehicles. In a refinement, at least one computing device within the network is operable to encode at least a portion of the animal data being provided to or by the at least one sensor, home station or the one or more unmanned aerial vehicles. The at least one computing device can include an unmanned aerial vehicle, a home station, a cloud server, an intermediary server, or other computing devices.

As set forth above, a user can pair one or more sensors to a single UAV within the control application, or pair one or more sensors to a plurality of UAVs or network of UAVs. Once the appropriate UAV is selected or a network of UAVs is selected, the user then enables a pairing function between a sensor and UAV or network of UAVs. This pairing function may initiate immediately if the sensor is in range of the one or more UAVs or initiate upon the sensor being in a range of the one or more UAVs. Alternatively, the sensor may transmit first to the control application (e.g., on home station 30) and then handoff to the UAV if the sensor is operable to pair with multiple transmission systems (e.g., broadcast network).

In a refinement, multiple UAVs can communicate with a single sensor. For example, if UAV1 is assigned to a designated area, and the targeted individual travels outside of that area, another UAV2 may communicate with the targeted individual once the individual is outside of the UAV1 designated area. In another refinement, multiple sensors may communicate with a single UAV (e.g., in the event, there are multiple targeted individuals using a single UAV or a single targeted individual is wearing multiple sensors). In another refinement, multiple UAVs can be utilized simultaneously and with the same one or more sensors, the multiple UAVs being operable to communicate with each other. For example, within a wireless mesh network, the UAV system may be operable to switch from one UAV to another UAV if the targeted subject moves out of range (e.g., assuming the UAV is responsible for covering a specific area and does not relocate), or other needs require it (e.g., bandwidth).

In another refinement, one or more unmanned aerial vehicles 20 can adjust then location, elevation, and/or transceiver positioning based upon the location of at least one sensor or the one or more targeted individuals. More specifically, the one or more UAVs can detect one or more characteristics of the at least one sensor or the one or more targeted individuals (e.g., location, positioning, signal strength), and the one or more UAVs can adjust their location or transceiver positioning based upon the location of the at least one sensor or one or more individuals. For example, if a UAV is tracking a group of targeted individuals, and the group of targeted individuals move to a new location, the UAV may change its location, adjust its elevation, and/or adjust its transceiver positioning to ensure optimal data transfer and collection between the at least one sensor and the UAV. In another refinement, the one or more UAVs may adjust its location, elevation, and/or UAV positioning based upon one or more tracking mechanisms for the one or more targeted individuals (e.g., optical hacking, sensor tracking). In a variation, the one or more UAVs may adjust its location, elevation, and/or positioning based upon the location of the sensors, which may be determined by one or more communication links. In another refinement, the UAV may handoff communication of the one or more sensors from the one or more targeted individuals to another one or more UAVs in the same network. In a variation for sporting applications, one or more UAVs can hover over a stadium (e.g., football, baseball, soccer) or racetrack and act as an intermediary computing device (e.g., transmission hub) for all targeted individuals (e.g., athletes, horses) and their associated sensors on a field of play. In other sporting applications, the UAV can track and follow a cycling race (and specifically the targeted participants), triathlons, marathons, and the like to collect data from one or more targeted individuals and take one or more actions upon the data (e.g., tag the data, manipulate the data, send the data to one or more endpoints). In a refinement, the elevation of the one or more UAVs may change or adjust based upon the location of the one or more targeted individuals. For example, a change in elevation by the one or more targeted individuals may cause the one or more UAVs to adjust its elevation. In a refinement, one or more unmanned aerial vehicles 20 can adjust their location, elevation, and/or transceiver positioning based upon one or more other factors (e.g., objects impeding line-of-sight, weather, an traffic, and the like). In another refinement, the one or more unmanned aerial vehicles 20 adjust their onboard one or more sensors (e.g., onboard camera sensors that change the zoom, focus, or location of where it is tracking). Adjustments may occur manually (e.g., remotely) or may be programmed to occur based upon one or more factors that may utilize one or more statistical modeling or artificial intelligence techniques.

In some variations, one or more unmanned aerial vehicles 20 may utilize one or more solutions for energy generation and conservation. Solutions may be focused on propulsion and/or sensor and system communication energy solutions (e.g., solutions focused on minimizing energy) expenditure related to data processing or other actions taken upon the data, signal acquisition, outbound data rate, UAV movement). Energy generation may include solar powered charging solutions, as well as solutions whereby the one or more UAVs come in contact with or communicate with, another device that provides energy to the one or more UAVs. Contact can include both physical contact and communicational contact (which may not be physical in nature). In a refinement, one or more unmanned aerial vehicles may be attached to, or in contact with, another object (e.g., an apparatus in a stadium or arena, another one or more UAVs, another computing device). This may be advantageous in the event the one or more UAVs utilize a mechanism for energy transfer with another object. The UAVs may also be connected via cable (e.g., ethernet, fiber) to one or more stationary objects (e.g., for faster connectivity, or to provide an energy supply).

In another refinement, UAV-based transmission system 10 and 10′ utilize one or more statistical modeling or artificial intelligence techniques such as machine learning methodologies to analyze animal data sets to create, modify, or enhance one or more predictions, probabilities, or possibilities. Given that machine learning-based systems are set up to learn from collected data rather than require explicit programmed instructions, its ability to search for and recognize patterns that may be hidden within one or more data sets enable machine learning-based systems to uncover insights from collected data that allow for one or more predictions to be made. Such predictions can be utilized for a wide variety of UAV and network functions including UAV location, formation, beam pattern, bandwidth management, energy management, home station functions, sensor functions. UAV deployment, and the like. Advantageously, because machine learning-based systems use data to learn, it oftentimes takes an iterative approach to improve model prediction and accuracy as new data enters the system, as well as improvements to model prediction and accuracy derived from feedback provided from previous computations made by the system (which also enables production of reliable and repeatable results).

In another refinement, the one or more unmanned aerial vehicles take one or more actions based upon one or more commands that are created, enhanced, or modified utilizing one or more artificial intelligence techniques. The one or more commands can be generated by the home station or the UAV. In another refinement, one or more unmanned aerial vehicle functions are optimized based upon one or more artificial intelligence techniques. Optimization can occur via the home station and/or the one or more UAVs. For example, the one or more home stations or UAVs utilize one or more artificial intelligence techniques (e.g., machine learning, deep learning) or statistical models for one or more actions or functions, including optimizing one or more beam patterns, transceiver locations (e.g., antenna locations), line of sight (e.g., positioning of the one or more UAVS to minimize packet loss from the one or more sensors on the one or more targeted individuals), beam widths, elevation (e.g., altitudes) for the one or more UAVs energy consumption of the one or more UAVs (e.g., power, fuel), UAV formation (e.g., three-dimensional formations) to optimize signal strength between sensor and UAV and coverage mapping, routing and movements (e.g., maximizing efficiency of any given route to minimize energy consumption), and the like. The use of one or more artificial intelligence techniques can also be used to optimize transmission signals (e.g., frequency of taking in data frequency of sending data to users, data quality), reduce network congestion, maximize the likelihood of a targeted detection or connection between the one or more UAVs and the one or more sensors based on a targeted individual’s location, and the like. In another refinement, one or more home station and/or sensor functions are optimized based upon one or more artificial intelligence techniques.

In another refinement, one or more UAVs may utilize one or more statistical modeling or artificial intelligence techniques to dynamically take one or more actions. The one or more actions may include adjustment of one or more sensor settings (e.g., the frequency at which the data is sampled or transmitted to the UAV), the frequency upon which the data is collected by the UAV (e.g., based upon energy considerations), UAV positioning and formation (e.g., based upon the locations of the one or more targeted individuals, weather), and the like. In a variation, the one or more UAVs may be operable to utilize one or more artificial intelligence techniques to evaluate the one or more biological sensor outputs (e.g., evaluating latency or signal strength between sensor and UAV), as well as conduct one or more data quality assessments. Based upon the evaluation or data quality assessment, the one or more UAVs may dynamically adjust its location, formation, trajectory, line of sight, beam pattern, transceiver locations (e.g., including its components such as one or more antennas), and the like.

In another refinement, artificial data may be generated utilizing one of more statistical models or artificial intelligence techniques, from which one or more simulations can be run to provide information that enables the one or more UAVs to take one or more actions. Based upon at least a portion of received sensor data from the one or more targeted individuals, the one or more UAVs may be operable to either provide (e.g., send) data to the home station, intermediary server, or third party system to run one or more simulations to generate simulated data. In a variation, the one or more UAVs may be operable to run one or more simulations to generate simulated data. Based upon the output from the one or more simulations, the one or more UAVs may take one or more actions. For example, the collected biological sensor data from the one or more targeted individuals, and non-animal data collected by the one or more UAVs, may trigger the one or more UAVs (or home station) to run one or more simulations related to the one or more targeted individuals, from which one or more predictions, probabilities, or possibilities may be calculated, computed, derived, extracted, exrapolated, simulated created, modified, enhanced, estimated, evaluated inferred, established, determined, deduced, observed, communicated, or actioned upon. The simulated data derived from at least a portion of the UAV-collected sensor data or its one or more derivatives can be used either directly or indirectly: (1) as a market upon which one or more wagers are placed or accepted; (2) to create, modify, enhance acquire, offer, or distribute one or more products; (3) to evaluate, calculate, derive, modify, enhance, or communicate one or more predictions, probabilities, or possibilities; (4) to formulate one or more strategies; (5) to take one or more actions; (6) to mitigate or prevent one or more risks; (7) as one or more readings utilized in one or more simulations, computations, or analyses; (8) as part of one or more simulations an output of which directly or indirectly engages with one or more users; (9) to recommend one or more actions; (10) as one or more core components or supplements to one or more mediums of consumption; (11) in one or more promotions; or (12) a combination thereof. For example, one or more simulations can be run related to the location of a group of targeted individuals to predict their expected location in order to position the one or more UAVs or network of UAVs (e.g., location, formation, elevation) to ensure optimal placement. In a refinement, simulations utilizing collected sensor data can also be used to predict targeted user’s movements, which can be based on the collected sensor data and one or more characteristics of the one or more targeted individuals (e.g., the activity the one or more targeted individuals are engaged in). Additional details related to an animal data prediction system with applications to sensor monitoring utilizing one or more unmanned aerial vehicles are disclosed in U.S. Pat. No. 62/833,970 filed Apr. 15, 2019, U.S Pat. No. 62/912,822 filed on Oct. 9, 2019; and U.S. Pat No. PCT/US20/28313 filed Apr. 15, 2020, the entire disclosures of which is hereby incorporated by reference.

In another refinement, one or more simulations are executed utilizing at least a portion of the received animal data to generate simulated data. In a variation, the one or more unmanned aerial vehicles, or the one or more computing devices in electionic communication with one or more unmanned aerial vehicles, execute one or more simulations. In another variation, one or more simulations utilize non-animal data as one or more inputs to generate simulated data. At least a portion of the simulated data, which is inclusive of its one or more derivatives, is utilized: (1) to create, enhance, or modify one or more insights or computed assets; (2) to create, modify enhance acquire, offer, or distribute one or more products; (3) to create evaluate, derive, modify, or enhance one or more predictions, probabilities, or possibilities; (4) to formulate one or more strategies; (5) to recommend one or more actions; (6) mitigate or prevent one or more risks; (7) as one or more readings utilized in one or more simulations, computations, or analyses; (8) as part of one or more simulations, an output of which directly or indirectly engages with one or more users; (9) as one or more core components or supplements to one or more mediums of consumption; (10) in one or more promotions; or (11) a combination thereof. In some cases, one or more simulations utilize one or more artificial intelligence or statistical modeling techniques to generate simulated data. In another variation, at least one computing device takes one or more actions based upon the simulated data. In a refinement, the at least one computing device is a home station, an intermediary server, a third-party computing device a cloud server, an unmanned aerial vehicle, or other computing devices.

In another refinement, one or more simulations incorporating collected sensor data can be run to predict a targeted individual’s or group of targeted individuals’ one or more anunal data readings (e.g., location, movements) and optimize the one or more UAVs. The one or more simulations can include collected sensor data, one or more characteristics of the one or more targeted individuals (e.g., the activity the one or more targeted individuals are engaged in), one or more types of non-animal data (e.g., weather search results or content from one or more mobile devices), and the like. For example, collecting location data from one or more targeted individuals to predict one or more movements via one or more simulations can enable efficiencies across the one or more UAVs including optimizing UAV formations (e.g., three-dimensional formations) to ensure optimal line of sight with the one or more targeted individuals, mapping of the UAVs, routing of the UAVs (e.g., maximizing efficiency of any given route to minimize energy consumption), sharing of data across UAVs and other computing devices (e.g., determining data may need to be shared or made available to other UAVs or computing devices vs. stored based upon the one or more predicted movements of the one or more targeted individuals, what information may need to be duplicated across UAVs to ensure a seamless handoff based on predicted movements, and the like), electronic communication between systems (e.g., maximizing the likelihood of a targeted detection or connection between the one or more UAVs and the one or more sensors based on a targeted individual’s location), antenna positioning, type of antenna utilized to communicate with one or more sensors or systems, antenna array positioning optimization of beam patterns and directions based upon predicted targeted individual location, placement/formation of the one or more UAVs based upon predicted targeted individual location (e.g., including projected changes in altitude, elevation), and the like. The one or more actions taken by the one or more UAVs upon the simulated data may result in an optimization of bandwidth (e.g., more available bandwidth), increased energy conservation for the one or more UAVs (e.g., enabling the UAV to utilize energy for additional functions or increased flight time), more reliable communication between sensor and UAV (e.g., stronger signal strength decreased data packet loss), maximization of coverage area and the like. Such simulations can occur on one or more UAVs, its associated cloud server, or within the network (e.g., via another computing device in communication with the one or more UAVs (e.g., home station, intermediary server).

In another refinement, one or more trained neural networks are utilized to generate simulated data (e.g., simulated animal data, other simulated data based upon the received animal data), the one or more trained neural networks having been trained with the received animal data or at least a portion thereof. In general, a neural network generates simulated animal data after being trained with real animal data. Animal data (e.g., ECG signals, heart rate, biological fluid readings) can be collected from one ot more sensors from one or more targeted individuals typically as a time series of observations. Sequence prediction machine learning algorithms can be applied to predict possible animal data values based on collected data. The collected animal data values will be passed on to one or more models during the training phase of the neural network. The neural network utilized to model this non-linear data set will train itself based on established principles of the one or more neural networks. More specifically, one or more neural networks may be trained with one or more of animal data sets to understand biological functions of a targeted individual and how one or more variables can affect any given biological function. The neural network can be further trained to understand what outcome (or outcomes) occurred based on the one or more biological functions and the impact ot the one or more variables, enabling correlative and causative analysis. For example, upon being trained to understand information such as the one or more biological functions of a targeted individual within any given scenario including a present scenario, the one or more variables that may impact the one or more biological functions of the targeted individual within any given scenario including a present scenario, the one or more outcomes that have previously occurred in any given scenario including a present scenario based on the one or more biological functions exhibited by the targeted individual and/or the one or more variables present, the one or more biological functions of individuals similar and dissimilar to the targeted individual in any given scenario including scenarios similar to a present scenario, the one or more other variables that may impact the one or more biological functions of the targeted individual in any given scenario including scenarios similar to a present scenario, the one or more variables that may impact the one or more biological functions of other individuals similar and dissimilar to targeted individual in any given scenario including scenarios similar to a present scenario, and the one or more outcomes that have previously occurred in any given scenario including scenarios similar to a present scenario based on the one or more biological functions exhibited by individuals similar and dissimilar to the targeted individual and/or the one or more variables, UAV-based transmission system 10 or 10′ may run one or more simulations to determine one or more predictions, probabilities, or possibilities.

The one or more types of neural networks can include, but are not limited to: Feedforward, Perceptron, Deep Feedforward, Radial Basis Network, Gated Recurrent Unit, Autoencoder (AE), Variational AE, Denoising AE, Sparse AE, Markov Chain, Hopfield Network, Boltzmann Machine, Restricted BM, Deep Belief Network, Deep Convolutional Network, Deconvolutional Network, Deep Convolutional Inverse Graphics Network, Liquid State Machine, Extreme Learning Machine, Echo State Network, Deep Residual Network, Kohonen Network, Support Vector Machine, Neural Turing Machine, Group Method of Data Handling, Probabilistic, Time delay, Convolutional, Deep Stacking Network, General Regression Neural Network, Self-Organizing Map, Learning Vector Quantization, Simple Recurrent, Reservoir Computing, Echo State, Bi-Directional, Hierarchal, Stochastic, Genetic Scale, Modular, Committee of Machines, Associative, Physical, Instantaneously Trained, Spiking Regulatory Feedback. Neocognitron, Compound Hierarchical-Deep Models, Deep Predictive Coding Network, Multilayer Kernel Machine, Dynamic, Cascading, Neuro-Fuzzy, Compositional Pattern Producing, Memory Networks, One-shot Associative Memory, Hierarchical Temporal Memory, Holographic Associative Memory, Semantic Hashing, Pointer Networks, and Encoder-Decoder Network.

Such methodologies can be applied to UAV-collected or UAV-based data (e.g., collected by the home station) to optimize UAV and network functions, including UAV location, formation, beam pattern, bandwidth management, energy management, and the like. In a variation, the one or more neural networks may be trained with multiple targeted individuals and one or more data sets from each targeted individual to more accurately generate a prediction, probability, or possibility. In another variation, one or more simulations may be run to first generate artificial data (e.g., artificial sensor data) based on real sensor data for each targeted individual (e.g., predict what their future heart rate beats per minute may look like on this upcoming run based on previous heart rate data, temperature, humidity, and other variables), and then utilize at least a portion of the generated artificial data (e.g., artificial sensor data) in one or more further simulations to determine the likelihood of any given outcome and/or make a prediction (e.g., the likelihood the targeted individual will experience heat stroke on the run). The present invention is not limited to the methodologies or types of neural networks utilized to generate artificial animal data from real animal data.

In another refinement, one or more actions can be taken based upon the collected animal data. For example, the one or more UAVs may detect or capture information that detects biological-based information based upon the one or more sensors (e.g., the targeted subject is experiencing a medical event such as a heart attack or a stroke), analyze the collected sensor data (e.g., utilizing one or more artificial intelligence techniques such as machine learning methods to find patterns within the data to generate predictive or probability-based information) or provide the data for analysis via another computing devices that accesses the data (e.g., via the cloud), and take one or more actions (e.g., send an alert to another system such as a hospital system notifying the system of such an alert; deliver one or more medications or drugs as a result of the UAV’s analysis of the one or more signals or readings; receive the analyzed information from the computing device providing analysis and send an alert to a third party). The action may be an alert that may include the one or more biological readings (e.g., a summary of the readings, location of the targeted individual from which the biological readings were captured) and/or other data (e.g., a predictive indicator communicating the likelihood a medical event will occur based on the collected information), along with information related to the one or more UAVs In a further refinement, the one or more UAVs may detect biological-based information that triggers the one or more UAVs to run one or more simulations, or triggers another computing device receiving or acquiring data from the one or more UAVs to run one or more simulations, from which one or more predictions, probabilities, or possibilities are derived (e.g., the collected biological sensor data provides readings that indicate abnormalities within the data that is associated with a specific medical episode, so the system runs one or more simulations to determine the likelihood that the targeted individual will experience the medical episode within n periods of time), and one or more actions are taken (e.g., the UAV may deliver a first-aid kit or other medical devices to aid m addressing the medical episode or send an alert to another system such as a hospital system or medical emergency system or send an alert to the targeted individual that a medical episode is about to occur). In another refinement, one or more UAVs may detect the animal data and another one or more UAVs may take the action (e.g., one UAV detects the biological data, another UAV runs the one or more simulations, another UAV interprets the captured sensor data and generated artificial information to protect the likelihood of a medical event occurring, and another UAV delivers the one or more drugs or prescriptions). In another refinement, one or more UAVs may detect the animal data and another one or more computing devices may take the action (e.g., the UAV captures the sensor data, sends the data to a third-party to run a simulation and deliver the appropriate drug or prescription based upon the output). Additional details related to systems for generating simulated animal data and models are disclosed in U.S. Pat. No. 62/897,064 filed Sep. 6, 2019, and U.S. Pat. No. 63/027,491 filed May 20, 2020, the entire disclosures of which is hereby incorporated by reference.

In another refinement, one or more artificial data values are generated. For example, in the event the one or more UAVs receive incomplete data (e.g., the sensor does not send all the data packets, the UAV docs not receive all the data packets, there are missing values from the one or more sensors or from the received data by the UAV, the set of data received by the UAV is noisy, or the UAV creates noise within the data), the home station, one or more UAVs, intermediary server, third-party system, or other computing devices (e.g., computing device 26) may utilize one or more techniques to fill-in missing values, or generate replacement values, with artificial data. Such techniques may also occur via the cloud server associated with the network. In many cases, the one or more sensors produce measurements (e.g., analog measurements such as raw AFE data) that are provided to a server, with a server applying methods or techniques to filter the data and generate one or more values (e.g., heart rate values). However, in cases where data has an extiemely low signal-to-noise ratio, or values are missing, pre-filter logic may be required to generate artificial data values. In one aspect, a pre-filter method whereby the system takes a number ot steps to “fix” the data generated from the sensor to ensure that the one or more data values generated are clean and fit within a predetermined range is proposed. The pre-filter logic, which can be housed on the one or more UAVs or its associated cloud, would ingest the data from the sensor detect any outlier or “bad” values replace these values with expected or “good” artificial values and pass along the “good” artificial values as its computation of the one or more biological data values (e.g., heart rate values). The term “fix” refers to an ability to create one or more alternative data values (i.e., “good” values) to replace values that may tall out of a pre-established threshold with the one or mote “good” data values aligning in the time series of genciated values and fitting within a pre-established threshold. These steps would occur prior to any logic taking action upon the received biological data to calculate the one or more biological data values (e.g., heart rate values).

Advantageously, the pre-filter logic and methodology for identification and replacement of one or more data values can be applied to any type of sensor data collected, including both raw and processed outputs received by the one or more UAVs. For illustration purposes, and while raw data such as analog measurements (AFE) can be converted into other waveforms such as surface electromyography (sEMG) signals, AFE conversion to ECG and heart rate (HR) values will be detailed. As previously described, the pre-filter logic becomes important in a scenario whereby the signal-to-noise ratio in the time series of generated AFE values from one or more sensors is at or close to zero, or numerically small. In this case, the previously described system and method to generate one or more heart rate values may ignore one or mote such values, which may result in no heart rate value genciated or a generated heart rate value that may fall outside the pre-established parameters, patterns and, or thresholds Such AFE values may result horn the subject taking an action that increases one or more other physiological parameters (e.g muscle activity), or in competing signals derived from the same sensor being introduced or deteriorating the connection, or from other variables. This in turn, may make for an inconsistent HR series.

To solve this problem, a method whereby one or more of one or more data values are created by looking at future values rather than previously generated values has been established. More specifically logic that is part of the one or more UAVs (e.g., onboarded on the one or more UAVs, in the cloud that is in communication with the one or more UAVs) may detect one or more outlier signal values and replace outlier values with one or more signal values that fall within an expected range (e.g., the established upper and lower bounds), thus having the effect of smoothing the series while at the same time decreasing the variance between each value. The established expected range may take into account a number of different variables including the individual the type of sensor, one or more sensor parameters, one or more of the sensor characteristics, one or more environmental factors, one or more characteristics of the individual activity of the individual, and the like. The expected range may also be created by one or more artificial intelligence or machine learning techniques that uses at least a portion of previously collected sensor data and/or its one or more derivatives, and possibly one or more of the aforementioned variables, to predict what an expected range may be. The expected range may also change over a period of time and be dynamic in nature, adjusting based on one or more variables (e.g., the activity the person is engaged in as environmental conditions). In a variation, one or more artificial intelligence techniques may be utilized, at least in part, to generate one or more artificial signal values within the expected range (e.g., upper and lower bound) derived from at least a portion of collected sensor data and/or its one or more derivatives from the one or more sensors.

To achieve the desired outcome of creating one or more values based upon future values, the system may first sample one or more of the sensor’s “normal” or “expected” AFE values and apply statistical tests and exploratory data analysis to determine the acceptable upper and lower bound of each AFE value generated by the sensor, which may include outlier detection techniques like interquartile range (IQR), distribution and percentile cut offs, kurtosis, and the like. A normal or expected AFE value may then be determined by utilizing at least a portion of previously collected sensor data. What is considered to be a normal or expected AFE value may also vary by sensor, by sensor parameter, or by other parameters/characteristics that may be factored into what is determined to be normal or expected (e.g., the subject, the activity the subject is engaged in).

Once an outlier is identified the pre-filter logic then uses a backward fill method to fill the one or more outliers (i.e., AFE values that fall outside of the accepted lower and upper bound) with the next value available that falls within the normal range in the current window of samples. This results in a cleaner and more predictable time-series of values which is devoid of un-processable noise. In a refinement, the one or more values are produced by utilizing artificial intelligence techniques (e.g., machine learning, deep learning) in which the model has been named to predict the next AFE value given a past sequence of AFE values, and/or as a replacement to one or more outliers in order to enable the sequence of values to fall within a normal range In a variation, a user could utilize a heuristic or mathematical formula-based method that describe waveforms similar to what an AFE signal produced from a sensor would be.

For heart rate values, the system may increase the amount of data used by the pre-filter logic processing the raw data to include n number of seconds worth of AFE data. An increase in the amount of data collected and utilized by the system enables the system to create a more predictable pattein of HR generated values as the number of intervals that are used to identify the QRS complex is increased. This occurs because HR is an average of the HR values calculated over one second sub-intervals. The n number of sec onds is a tunable parameter that may be pre-determined or dynamic. In a refinement, artificial intelligence techniques may be utilized to predict the n number of seconds of AFE data required to generate one or more values that fall within a given range based on one or more previously collected data sets.

While the pre-processing of the data may not replicate the possible R-peaks in a QRS complex, the pulling in of one or more noisy values into the range of a normal or expected signal allows the downstream filter and system generating the HR values to produce one or mote HR values that fall within the expected range in the absence of a quality signal. Additional details related to a system for measuring a heart rate and other biological data are disclosed in U.S. Pat. Application No. 16/246,921 filed Jan. 14, 2019 and U.S. Pat. No. PCT/US20/13461 filed Jan. 14, 2020; the entire disclosures of which are hereby incorporated by reference.

In a refinement, the logic contained within the one or more UAVs generates artificial data values to complete a data set. For example, a sensor that is collecting any given biological data (e.g., heart rate) may have an occurrence that prevents the sensor from collecting, analyzing and/or distributing data to the simulation (e.g., the one or more sensors fall off the subject, stops collecting data because it runs out of power, and the like). In this example, the one or more UAVs (or other computing devices such as an intermediary server) can include logic to run one or more simulations to create one or more artificial data sets to complete the data set (e.g., if a subject is on a 40 minute run and the heart rate sensor runs out of battery after 30 minutes, the simulation system can generate the final 10 minutes of heart rate data, which may take into account more of more variables including previously collected data and data sets, speed, distance, environmental conditions, and the like).

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally the features of various implementing embodiments may be combined to form further embodiments of the invention.

What is claimed is:

1. An unmanned aerial vehicle-based data collection and distribution system comprising:
a source of animal data that is electronically transmittable, the source of animal data including at least one sensor, animal data being collected from at least one targeted individual;
an unmanned aerial vehicle that receives the animal data from the source of animal data as a first set of received animal data, the unmanned aerial vehicle having a transceiver operable to receive one or more signals from the source of animal data and to send one or more control signals to the source of animal data; and
a computing device that is operable to receive at least a portion of the first set of received animal data.

2. The system of claim 1 wherein the animal data is human data.

3. The system of claim 1 wherein the computing device is at least one of: a home station, an intermediary server, a third-party computing device, a cloud server, another unmanned aerial vehicle, or other computing devices.

4. The system of claim 1 wherein the unmanned aerial vehicle takes one or more actions utilizing received animal data.

5. The system of claim 4 wherein the one or more actions are selected from the group consisting of: normalizing the animal data, associating a timestamp with the animal data, aggregating the animal data, applying a tag to the animal data, storing the animal data, manipulating the animal data, processing the data, denoising the animal data, enhancing the animal data, organizing the animal data, analyzing the animal data, anonymizing the animal data, visualizing the animal data, synthesizing the animal data, summarizing the animal data, synchronizing the animal data, replicating the animal data, displaying the animal data, distributing the animal data, productizing the animal data, performing bookkeeping on the animal data, or combinations thereof.

6. The system of claim 4 wherein the one or more actions includes at least one coordinated action with another computing device upon the same set of received animal data.

7. The system of claim 1 wherein the unmanned aerial vehicle is operable to send animal data to another computing device.

8. The system of claim 1 wherein the unmanned aerial vehicle attaches metadata to the animal data.

9. The system of claim 8 wherein the metadata includes one or more characteristics related to the at least one targeted individual, the at least one sensor, the unmanned aerial vehicle, the animal data, or combination thereof.

10. The system of claim 1 wherein if the unmanned aerial vehicle is not in electronic communication with the at least one sensor, the unmanned aerial vehicle is operable to initiate electronic communication with the at least one sensor after one or more of the following parameter changes: time, one or more characteristics of the at least one sensor, one or more characteristics of the at least one targeted individual, or one or more characteristics of one or more unmanned aerial vehicles.

11. The system of claim 1 wherein one or more electronic communications between the at least one sensor and a home station is transferred from a non-unmanned aerial vehicle computing device to one or more unmanned aerial vehicles, or vice versa.

12. The system of claim 1 wherein a network consisting of at least one home station, at least one sensor, and at least one unmanned aerial vehicle is operable to monitor one or more characteristics related to the at least one sensor, the at least one unmanned aerial vehicle, electronic communication within the network, collected animal data, distribution of the collected animal data, or a combination thereof.

13. The system of claim 12 wherein the network includes one or more intermediary servers, third-party computing devices, cloud servers, or combinations thereof.

14. The system of claim 12 wherein two or more unmanned aerial vehicles operate within the network, with one or more home stations operable to electronically communicate with the two or more unmanned aerial vehicles as part of the network, and two or more unmanned aerial vehicles operable to electronically communicate with each other.

15. The system of claim 14 wherein electronic communication includes providing animal data from one unmanned aerial vehicle to another one or more unmanned aerial vehicles.

16. The system of claim 14 wherein the two or more unmanned aerial vehicles execute one or more coordinated actions in response to one or more commands.

17. The system of claim 12 wherein at least one computing device within the network is operable to encode animal data being provided to or by the at least one sensor, home station, or the unmanned aerial vehicle.

18. The system of claim 1 wherein a home station is programmed to select one or more unmanned aerial vehicles, or network that includes one or more unmanned aerial vehicles, to connect with the at least one sensor based on one or more of the following characteristics: unmanned aerial vehicle location, unmanned aerial vehicle coverage, unmanned aerial vehicle payload, unmanned aerial vehicle bandwidth, network coverage, network payload, network bandwidth, targeted individual location, sensor location, an energy constraint, signal strength, an environmental condition, and signal quality.

19. The system of claim 18 wherein the home station or the one or more unmanned aerial vehicles provide one or more commands to the at least one sensor to take one or more actions based upon information derived from, at least in part, one or more characteristics of the at least one sensor.

20. The system of claim 1 wherein one or more unmanned aerial vehicles are attached to, or in contact with, another object.

21. The system of claim 1 wherein the unmanned aerial vehicle is operable to perform at least one functionality of a home station, intermediary server, or cloud server.

22. The system of claim 1 wherein one or more unmanned aerial vehicles have attached to, affixed to, integrated with, or embedded within, at least one sensor that captures one or more signals or readings.

23. The system of claim 22 wherein at least a portion of the one or more signals or readings are provided by the one or more unmanned aerial vehicles to another computing device.

24. The system of claim 22 wherein at least a portion of the one or more signals or readings are used by the one or more unmanned aerial vehicles to transform collected animal data into one or more computed assets or insights.

25. The system of claim 1 wherein the unmanned aerial vehicle is operable to electronically communicate with the at least one sensor from one or more targeted individuals using one or more wireless communication protocols.

26. The system of claim 1 wherein the unmanned aerial vehicle take one or more actions in response to one or more calculations, computations, predictions, probabilities, possibilities, estimations, evaluations, inferences, determinations, deductions, observations, or forecasts that are derived from, at least in part, the first set of received animal data.

27. The system of claim 26 wherein one or more artificial intelligence or statistical modeling techniques are utilized to create, enhance, or modify the one or more actions taken by the unmanned aerial vehicle.

28. The system of claim 26 wherein the one or more actions include at least one of: providing one or more alerts to one or more computing devices or providing animal data to one or more computing devices that generate one or more alerts based upon the animal data.

29. The system of claim 28 wherein one or more actions are taken by one or more unmanned aerial vehicles or computing devices in response to the one or more alerts.

30. The system of claim 29 wherein one or more artificial intelligence or statistical modeling techniques are utilized to create, enhance, or modify one or more actions taken by the one or more unmanned aerial vehicles or computing devices.

31. The system of claim 26 wherein the one or more calculations, computations, predictions, probabilities, possibilities, estimations, evaluations, inferences, determinations, deductions, observations, or forecasts include at least a portion of non-animal data.

32. The system of claim 1 wherein at least one of the unmanned aerial vehicle, home station, intermediary server, cloud server, or other computing device are operable to assign one or more classifications to the animal data, the one or more classifications including at least one of: computed asset classifications, insight classifications, targeted individual classifications, sensor classifications, unmanned aerial vehicle classifications, data property classifications, data timeliness classifications, or data context classifications.

33. The system of claim 1 wherein the at least one sensor and/or one or more appendices of the at least one sensor are affixed to, are in contact with, or send one or more electronic communications in relation to or derived from, a targeted individual’s body, skin, eyeball, vital organ, muscle, hair, veins, biological fluid, blood vessels, tissue, or skeletal system, embedded in a targeted individual, lodged or implanted in the at least one targeted individual, ingested by the targeted individual, integrated to include at least a portion of the targeted individual, or integrated as part of, or affixed to or embedded within, a fabric, textile, cloth, material, fixture, object, or apparatus that contacts or is in electronic communication with the targeted individual either directly or via one or more intermediaries.

34. The system of claim 33 wherein the at least one sensor is a biosensor that gathers physiological, biometric, chemical, biomechanical, location, environmental, genetic, genomic, or other biological data from one or more targeted individuals.

35. The system of claim 33 wherein the at least one sensor is configured to gather or derive at least one of: facial recognition data, eye tracking data, blood flow data, blood volume data, blood pressure data, biological fluid data, body composition data, biochemical composition data, biochemical structure data, pulse data, oxygenation data, core body temperature data, skin temperature data, galvanic skin response data, perspiration data, location data, positional data, audio data, biomechanical data, hydration data, heart-based data, neurological data, genetic data, genomic data, skeletal data, muscle data, respiratory data, kinesthetic data, thoracic electrical bioimpedance data, ambient temperature data, humidity data, barometric pressure data, or elevation data.

36. The system of claim 1 wherein the computing device executes a control application that provides one or more commands to the unmanned aerial vehicle, the one or more commands initiating at least one of the following actions: (1) activating one or more sensors; (2) streaming at least a portion of collected animal data to one or more computing devices; (3) selecting one or more data streams to be sent to one or more computing devices; (4) selecting a frequency upon which animal data is sent to one or more computing devices; (5) taking one or more actions upon collected animal data, and sending actioned upon data to one or more computing devices; (6) changing or adjusting one or more settings within the at least one sensor; (7) taking one or more actions by the at least one sensor; (8) changing or adjusting one or more characteristics of one or more unmanned aerial vehicles; (9) taking one or more actions based upon information derived from the animal data; (10) providing electronic communication support between one or more home stations and one or more other computing devices; or (11) storing sensor data.

37. The system of claim 1 wherein the unmanned aerial vehicle includes a data acquisition unit that electronically communicates with the at least one sensor.

38. The system of claim 37 wherein the data acquisition unit includes a transceiver module operable to send one or more commands to the at least one sensor and receive one or more data signals or readings from the at least one sensor.

39. The system of claim 38 wherein the transceiver module is operable to electronically communicate with one or more computing devices.

40. The system of claim 37 wherein the data acquisition unit includes a microprocessor operable to execute one or more data processing steps.

41. The system of claim 40 wherein the data acquisition unit includes memory module and an input/output module in electronic communication with the microprocessor.

42. The system of claim 37 wherein the data acquisition unit includes a communication module.

43. The system of claim 1 wherein the unmanned aerial vehicle is operable to electronically communicate with two or more sources of animal data simultaneously.

44. The system of claim 1 wherein two or more unmanned aerial vehicles are operable to electronically communicate with the same source of animal data.

45. The system of claim 1 wherein one or more simulations are executed utilizing animal data to generate simulated data.

46. The system of claim 45 wherein at least a portion of the simulated data is utilized: (1) to create, enhance, or modify one or more insights or computed assets; (2) to create, modify, enhance, acquire, offer, or distribute one or more products; (3) to create, evaluate, derive, modify, or enhance one or more predictions, probabilities, or possibilities; (4) to formulate one or more strategies; (5) to recommend one or more actions; (6) mitigate or prevent one or more risks; (7) as one or more readings utilized in one or more simulations, computations, or analyses; (8) as part of one or more simulations, an output of which directly or indirectly engages with one or more users; (9) as one or more core components or supplements to one or more mediums of consumption; (10) in one or more promotions; or (11) a combination thereof.

47. The system of claim 45 wherein the one or more simulations utilize one or more artificial intelligence or statistical modeling techniques to generate simulated data.

48. The system of claim 47 wherein one or more trained neural networks are utilized to generate simulated data, the one or more trained neural networks having been trained with received animal data.

49. The system of claim 45 wherein one or more unmanned aerial vehicles, or the one or more computing devices in electronic communication with the one or more unmanned aerial vehicles, execute the one or more simulations.

50. The system of claim 45 wherein the one or more simulations utilize non-animal data as one or more inputs to generate simulated data.

51. The system of claim 45 wherein at least one computing device takes one or more actions based upon the simulated data.

52. The system of claim 51 wherein the at least one computing device is a home station, an intermediary server, a third-party computing device, a cloud server, the unmanned aerial vehicle, or other computing device.
